SEQUENCE LISTING (3 sequences)

SEQ ID NO: 1 - protein, 304 aa, Paenibacillus hunanensis
ATGTGKGVLGDTKSFTVGTSGSSYVMTDSTRGKGIQTYTASNRTSLPGSTVTSSSSTFNDPASVDAHAYAQKVYDFYKSNFNRNSIDGNGLAIRSTTHYSTRYNNAFWNGSQMVYGDGDGSQFIAFSGDLDVVGHELTHGVTEYTANLEYYGQSGALNESISDIFGNTIEGKNWMVGDAIYTPGVSGDALRYMDDPTKGGQPARMADYNNTSADNGGVHTNSGIPNKAYYLLAQGGTFGGVNVTGIGRSQAIQIVYRALTYYLTSTSNFSNYRSAMVQASTDLYGANSTQTTAVKNSLSAVGIN

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 484 aa, Bacillus amyloliquefaciens
AVNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

(19) United States
(12) Patent Application Publication
(10) Pub. No.: US 2026/0365947 A1
(43) Pub. Date: Nov. 19, 2026

(54) WATER-SOLUBLE UNIT DOSE SOLID DETERGENT ARTICLE COMPRISING PROTEASES

(71) Applicant: THE PROCTER & GAMBLE COMPANY, Cincinnati, OH (US)

(72) Inventors: Lindsey HEATHCOTE, Northumberland (GB); Michelle JACKSON, Newcastle upon Tyne (GB); Mark Robert SIVIK, Mason, OH (US); Philip Frank SOUTER, Morpeth Northumberland (GB)

(21) Appl. No.: 19/668,952

(22) Filed: May 6, 2026

(30) Foreign Application Priority Data
May 9, 2025 (WO) PCT/US2025/028539

(51) Int. Cl. C11D 17/04; C11D 1/14; C11D 1/22; C11D 1/72; C11D 1/83; C11D 3/37; C11D 3/386

(52) U.S. Cl. CPC C11D 17/044; C11D 1/83; C11D 3/3707; C11D 3/3753; C11D 3/38609; C11D 1/146; C11D 1/22; C11D 1/72

(57) ABSTRACT

A water-soluble unit-dose solid detergent article including a porous or a fibrous structure, the article includes a surfactant system, a serine protease and a metalloprotease variant, the variant having a sequence identity of at least 60% to the metalloprotease of SEQ ID NO:1.

### TECHNICAL FIELD

The present disclosure relates to a water-soluble unit-dose solid detergent article comprising a porous or a fibrous structure. The water-soluble unit-dose solid detergent article comprises a surfactant system, a serine protease and a metalloprotease. It also relates to method of using said solid article.

### REFERENCE TO AN ELECTRONIC SEQUENCE LISTING

This application contains a Sequence Listing in computer readable form. The contents of the electronic sequence listing (CM05862_Sequence.xml; Size: 4,445 bytes; and Date of Creation: 17 Mar. 2026) is herein incorporated by reference in its entirety.

### BACKGROUND

Water-soluble unit dose articles are desired by consumers as they provide a convenient, efficient and clean way of dosing a fabric treatment composition. Water-soluble unit dose articles provide a measured dosage of a treatment composition, thereby avoiding over or under dosing. Fibrous water-soluble unit dose articles are of increasing interest to consumers. Porous water-soluble unit dose articles are also of increasing interest to consumers. The technology related to such articles continues to advance in terms of providing the desired active agents with the articles enabling the consumers to do the job that they wish to accomplish. Consumers desire solid fibrous or porous water-soluble unit dose articles that clean as well or better than conventional forms of fabric treatment compositions, such as liquids, powders and unit dose articles constructed of water-soluble films. In comparison with traditional granular or liquid detergents in the same product category, such detergent articles can have better structural integrity, are more concentrated and easier to store, ship/transport, carry, and handle. Such detergent articles seem to have good sensory appeal to the consumers, in particular when used in laundry.

During the laundry process, the pH of the wash water can change significantly due to the introduction of soil and contaminants from the soiled fabrics. As the fabrics release dirt, oils, and organic materials, these substances can interact with the wash water, altering its chemical composition. For instance, many types of soil, such as sweat or food residues, can be acidic, which may lower the pH of the wash water. Conversely, certain detergents and additives used in laundry are often alkaline, which can raise the pH of the wash water. This dynamic interaction between the soil and the wash water not only influences the effectiveness of the cleaning agents but also impacts the overall washing performance, as the pH level plays a crucial role in the solubility of stains and the activity of actives in the detergent.

Water-soluble unit-dose solid articles may result in detersive products with sub-optimal or less desirable proteinaceous stain removal. The pH modification can negatively impact protease performance.

There is therefore a need for water-soluble unit-dose solid articles that provide improved proteinaceous stain removal across a broad pH range.

### SUMMARY

A water-soluble unit dose solid detergent article is disclosed. The water-soluble unit dose solid article comprises a porous or a fibrous structure. It also comprises a surfactant system, a serine protease and a metalloprotease. There is also disclosed a method of cleaning a surface, preferably a fabric, using said water-soluble unit dose solid detergent article.

### BRIEF DESCRIPTION OF THE DRAWINGS

FIG. 1 is a graph for examples described herein.

FIG. 2 is a graph for examples described herein.

### DETAILED DESCRIPTION

### Definitions

Features and benefits of the present disclosure will become apparent from the following description, which includes examples intended to give a broad representation of the detergent article. Various modifications will be apparent to those skilled in the art from this description and from practice of the present products and methods. The scope is not intended to be limited to the particular forms disclosed and this disclosure covers all modifications, equivalents, and alternatives falling within the spirit and scope of the present products and methods as defined by the claims.

The term “water-soluble” as used herein refers to the ability of a sample material to completely dissolve in or disperse into water leaving no visible solids or forming no visibly separate phase, when at least about 25 grams, preferably at least about 50 grams, more preferably at least about 100 grams, most preferably at least about 200 grams, of such material is placed in one liter (1 L) of deionized water at 20 °C and under the atmospheric pressure with sufficient stirring.

As used herein, the articles including “the,” “a” and “an” when used in a claim or in the specification, are understood to mean one or more of what is claimed or described.

As used herein, the terms “include,” “includes” and “including” are meant to be non-limiting.

The term “substantially free of” or “substantially free from” as used herein refers to either the complete absence of an ingredient or a minimal amount thereof merely as impurity or unintended byproduct of another ingredient. A composition that is “substantially free” of/from a component means that the composition comprises less than about 0.5%, 0.25%, 0.1%, 0.05%, or 0.01%, or even 0%, by weight of the composition, of the component.

It should be understood that the term “comprise” includes also embodiments where the term “comprises” means “consists of” or “consists essentially of.”

The term “open celled foam” or “open cell pore structure” as used herein refers to a solid, interconnected, polymer-containing matrix that defines a network of spaces or cells that contain a gas, typically a gas (such as air), without collapse of the foam structure during the drying process, thereby maintaining the physical strength and cohesiveness of the solid. The interconnectivity of the structure may be described by a Percent Open Cell Content, which is measured by Test 3 disclosed in WO 2024/147008 A1, page 44.

All cited patents and other documents are, in relevant part, incorporated by reference as if fully restated herein. The citation of any patent or other document is not an admission that the cited patent or other document is prior art with respect to the present disclosure.

In this description, all concentrations and ratios are on a weight basis of the composition unless otherwise specified.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Water-Soluble Unit-Dose Solid Article

The water-soluble unit-dose solid article comprises a porous or a fibrous structure, the structure can be formed by one more sheet(s). In the case of more than one sheet. The sheets are stacked or bonded together to form the final product. These articles are designed to dissolve in water, releasing the detergent contents for cleaning purposes.

Each sheet can contribute to the overall strength, solubility, and functionality of the detergent article. For instance, multiple sheets may be used to control the rate of dissolution or to encapsulate different ingredients that may need to be released at various stages of the washing process.

The water-soluble unit-dose solid detergent article can have a non-fibrous and porous structure and characterized by a unique open cell foam (OCF) structure (as described hereinafter), which allows for incorporation of a high surfactant content while still providing fast dissolution. Consequently, highly concentrated cleaning compositions can be formulated into the solid sheet articles of the present disclosure to provide a new and superior cleansing experience to the consumers. Preferably, the solid sheet article of the present disclosure comprises from about 30% to about 80%, preferably from about 40% to about 70%, more preferably from about 50% to about 65%, of surfactants by total weight of said solid sheet. The process for making the sheet is described in WO2020/147008 A1, pages 28 to 37.

The water-soluble unit-dose solid detergent article can have a fibrous structure The fibrous water-soluble unit dose article may include any of the disclosures found in U.S. patent application Ser. No. 15/880,594 filed on Jan. 26, 2018; U.S. patent application Ser. No. 15/880,599 filed Jan. 26, 2018; and U.S. patent application Ser. No. 15/880,604 filed Jan. 26, 2018; incorporated by reference in their entirety. The water-soluble unit-dose solid detergent article can be in the form of a pouch made from a water-soluble non-woven fibrous sheet as described in EP 4 389 866A1.

These fibrous water-soluble unit dose articles can be dissolved under various wash conditions, e.g., low temperature, low water and/or short wash cycles or cycles where consumers have been overloading the machine, especially with items having high water absorption capacities, while providing sufficient delivery of active agents for the intended effect on the target consumer substrates (with similar performance as today's liquid products). Furthermore, the water-soluble unit dose articles described herein can be produced in an economical manner by spinning fibers comprising active agents. The water-soluble unit dose articles described herein also have improved cleaning performance.

The surface of the water-soluble unit dose article may comprise a printed area. The printed area may cover between about 10% and about 100% of the surface of the article. The area of print may comprise inks, pigments, dyes, bluing agents or mixtures thereof. The area of print may be opaque, translucent or transparent. The area of print may comprise a single color or multiple colors. The printed area maybe on more than one side of the article and contain instructional text and/or graphics. The surface of the water-soluble unit dose article may comprise an aversive agent, for example a bittering agent. Suitable bittering agents include, but are not limited to, naringin, sucrose octacetate, quinine hydrochloride, denatonium benzoate, or mixtures thereof. Any suitable level of aversive agent may be used. Suitable levels include, but are not limited to, 1 to 5000 ppm, or even 100 to 2500 ppm, or even 250 to 2000 ppm.

The water-soluble unit dose articles disclosed herein can comprise a water-soluble fibrous or porous structure and one or more particles. The water-soluble fibrous structure may comprise a plurality of fibrous elements, for example a plurality of filaments. The one or more particles, for example one or more active agent-containing particles, may be distributed throughout the structure. The water-soluble unit dose article may comprise a plurality of two or more and/or three or more fibrous elements that are inter-entangled or otherwise associated with one another to form a fibrous structure and one or more particles, which may be distributed throughout the fibrous structure.

The fibrous water-soluble unit dose articles may exhibit a thickness of greater than 0.01 mm and/or greater than 0.05 mm and/or greater than 0.1 mm and/or to about 100 mm and/or to about 50 mm and/or to about 20 mm and/or to about 10 mm and/or to about 5 mm and/or to about 2 mm and/or to about 0.5 mm and/or to about 0.3 mm as measured by the Thickness Test Method described herein.

The fibrous water-soluble unit dose articles may have basis weights of from about 500 grams/m² to about 5,000 grams/m², or from about 1,000 grams/m² to about 4,000 grams/m², or from about 1,500 grams/m² to about 3,500 grams/m², or from about 2,000 grams/m² to about 3,000 grams/m², as measured according to the Basis Weight Test Method described herein.

The fibrous water-soluble unit dose article may comprise a water-soluble fibrous structure and a plurality of particles distributed throughout the structure, where the water-soluble fibrous structure comprises a plurality of identical or substantially identical, from a compositional perspective, fibrous elements. The water-soluble fibrous structure may comprise two or more different fibrous elements. Non-limiting examples of differences in the fibrous elements may be physical differences, such as differences in diameter, length, texture, shape, rigidness, elasticity, and the like; chemical differences, such as crosslinking level, solubility, melting point, Tg, active agent, filament-forming material, color, level of active agent, basis weight, level of filament-forming material, presence of any coating on fibrous element, biodegradable or not, hydrophobic or not, contact angle, and the like; differences in whether the fibrous element loses its physical structure when the fibrous element is exposed to conditions of intended use; differences in whether the fibrous element's morphology changes when the fibrous element is exposed to conditions of intended use; and differences in rate at which the fibrous element releases one or more of its active agents when the fibrous element is exposed to conditions of intended use. Two or more fibrous elements within the fibrous structure may comprise different active agents. This may be the case where the different active agents may be incompatible with one another, for example an anionic surfactant and a cationic polymer. When using different fibrous elements, the resulting structure may exhibit different wetting, imbibition, and solubility characteristics.

The fibrous water-soluble unit dose article may exhibit different regions, such as different regions of basis weight, density, caliper, and/or wetting characteristics. The fibrous water-soluble unit dose article may be compressed at the point of edge sealing. The fibrous water-soluble unit dose article may comprise texture on one or more of its surfaces. A surface of the fibrous water-soluble unit dose article may comprise a pattern, such as a non-random, repeating pattern. The fibrous water-soluble unit dose article may comprise apertures. The fibrous water-soluble unit dose article may comprise a fibrous structure having discrete regions of fibrous elements that differ from other regions of fibrous elements in the structure. The fibrous water-soluble unit dose article may be used as is or it may be coated with one or more active agents.

The fibrous water-soluble unit dose article may comprise one or more plies. The fibrous water-soluble unit dose article may comprise at least two and/or at least three and/or at least four and/or at least five plies. The fibrous plies can be fibrous structures. Each ply may comprise one or more layers, for example one or more fibrous element layers, one or more particle layers, and/or one or more fibrous element/particle mixture layers. The layer(s) may be sealed. In particular, particle layers and fibrous element/particle mixture layers may be sealed, such that the particles do not leak out. The water-soluble unit dose articles may comprise multiple plies, where each ply comprises two layers, where one layer is a fibrous element layer and one layer is a fibrous element/particle mixture layer, and where the multiple plies are sealed (e.g., at the edges) together. Sealing may inhibit the leakage of particles as well as help the unit dose article maintain its original structure. However, upon addition of the water-soluble unit dose article to water, the unit dose article dissolves and releases the particles into the wash liquor.

The fibrous elements and/or particles may be arranged within the water-soluble unit dose article, in a single ply or sheet, or in multiple plies or sheets, to provide the article with two or more regions that comprise different active agents. For example, one region of the article may comprise bleaching agents and/or surfactants and another region of the article may comprise softening agents.

The fibrous water-soluble unit dose article can be viewed hierarchically starting from the form in which the consumer interacts with the water-soluble article and working backward to the raw materials from which the water-soluble article is made, e.g., plies, fibrous structures, and particles. The fibrous plies can be fibrous structures.
Surprisingly, it has been found that water-soluble unit-dose solid detergent article comprising a porous or a fibrous structure and
   a surfactant system
   a serine protease; and
   a metalloprotease having a sequence identity of at least 60%, preferably at least 80%, more preferably at least 90%, more preferably at least 95%, more preferably at least 98% to the metalloprotease of SEQ ID NO:1 (SEQ ID NO: 33 of WO2014/194034) provides very good proteinaceous soil removal across a broad pH range of the wash liquor.

Preferred water-soluble unit dose solid detergent articles can comprise water-soluble fibrous or non-fibrous porous structures and preferably one or more particles comprising detergent actives. More specifically, water-soluble unit dose articles described herein may comprise a water-soluble fibrous structure and preferably one or more rheology-modified particles comprising: (a) from about 10 wt % to about 80 wt % of an anionic surfactant; and (b) from about 0.5 wt % to about 20 wt % of a rheology modifier. The particles described herein may comprise one or more additional active agents (in addition to surfactant as described hereinabove).

The rheology-modified particle may comprise:
(a) from about 10 wt % to about 80 wt % of anionic surfactant;
(b) from about 0.5 wt % to about 20 wt % of a rheology modifier selected from the group consisting an alkoxylated amine, preferably an alkoxylated polyamine, more preferably a quaternized or non-quaternized alkoxylated polyethyleneimine, wherein said alkoxylated polyalkyleneimine has a polyalkyleneimine core with one or more alkoxy side chains bonded to at least one nitrogen atom in the polyalkyleneimine core, an ethylene oxide-propylene oxide-ethylene oxide (EOx₁POyEOx₂) triblock copolymer wherein each of x₁ and x₂ is in the range of about 2 to about 140 and y is in the range of from about 15 to about 70, and mixtures thereof.

As used herein, the term “rheology modifier” means a material that interacts with concentrated surfactants, preferably concentrated surfactants having a mesomorphic phase structure, in a way that substantially reduces the viscosity and elasticity of said concentrated surfactant in the presence of solvents such as water. Suitable rheology modifiers include, but are not limited to, sorbitol ethoxylate, glycerol ethoxylate, sorbitan esters, tallow alkyl ethoxylated alcohol, ethylene oxide-propylene oxide-ethylene oxide (EOx₁POyEOx₂) triblock copolymers wherein each of x₁ and x₂ is in the range of about 2 to about 140 and y is in the range of from about 15 to about 70, alkoxylated amines, alkoxylated polyamines, polyethyleneimine (PEI), alkoxylated variants of PEI, and preferably ethoxylated PEI, and mixtures thereof. The rheology modifier may comprise one of the polymers described above, for example, ethoxylated PEI, in combination with a polyethylene glycol (PEG) having a weight average molecular weight of about 2,000 Daltons to about 8,000 Daltons.

As used herein, the term “functional rheology modifier” means a rheology modifier that has additional detergent functionality. In some cases, a dispersant polymer, described herein below, may also function as a functional rheology modifier. A functional rheology modifier may be present in the detergent particles of the present product at a level of from about 0.5% to about 20%, preferably from about 1% to about 15%, more preferably from about 2% to about 10% by weight of the composition.

Without being limited by theory, it is believed that functional rheology modifiers are able to interact with the molecular structure of intermediate-phase surfactants, especially alcohol-based anionic sulfate surfactants, said intermediate phases having more water than solid-phase surfactant, and less water than micellar phases typical of wash solutions. In other words, intermediate phase surfactants represent a transitional state from solid to micellar phase that may be achieved in the successful use of fibrous water-soluble unit dose articles comprising a water-soluble fibrous structure and particles; if the rheology of this intermediate state is too viscous or sticky, it may under circumstances of insufficient local dilution and/or insufficient shear result in undesired residue on fabrics. By substantially reducing the viscosity and elasticity of said intermediate phases, rheology modifiers aid dispersion, mitigating the risk of forming residue on fabrics. Further, for any residue, e.g., lump-gels, that may form, rheology modifiers can reduce their persistence. The net effect is to mitigate the occurrence of surfactant residues that persist on fabrics through the wash.

Alkoxylated Amine: The alkoxylated amine may be partially or fully protonated or not protonated across the pH range of the concentrated surfactant mixture. Alternatively, the alkoxylated amine may be partially or fully quaternized. The alkoxylated amine may be non-quaternized. The alkoxylated amine may comprise ethoxylate (EO) groups.

The alkoxylated amine may be linear, branched, or combinations thereof, preferably branched.

The alkoxylated amine may contain two or more amine moieties, such as N,N,N′,N′-tetra(2-hydroxyethyl)ethylenediamine (also described as a type of hydroxylalkylamine). N,N,N′,N′-tetra(2-hydroxyethyl)ethylenediamine also functions as a chelant.

The alkoxylated amine may comprise (or be) an alkoxylated amine comprises an alkoxylated polyalkyleneimine. The alkoxylated polyalkyleneimine may be an alkoxylated polyethyleneimine (PEI).

Typically, the alkoxylated polyalkyleneimine polymer comprises a polyalkyleneimine backbone. The polyalkyleneimine may comprise C2 alkyl groups, C3 alkyl groups, or mixtures thereof, preferably C2 alkyl groups. The alkoxylated polyalkyleneimine polymer may have a polyethyleneimine (“PEI”) backbone.

The alkoxylated PEI may comprise a polyethyleneimine backbone having a weight average molecular weight of from about 400 to about 1000, or from about 500 to about 750, or from about 550 to about 650, or about 600, as determined prior to ethoxylation.

The PEI backbones of the polymers described herein, prior to alkoxylation, may have the general empirical formula:

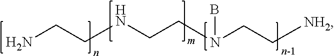

where B represents a continuation of this structure by branching. In some aspects, n+m is equal to or greater than 8, or 10, or 12, or 14, or 18, or 22.

The alkoxylated polyalkyleneimine polymer comprises alkoxylated nitrogen groups. The alkoxylated polyalkyleneimine polymer may independently comprise, on average per alkoxylated nitrogen, up to about 50, or up to about 40, or up to about 35, or up to about 30, or up to about 25, or up to about 20, alkoxylate groups. The alkoxylated polyalkyleneimine polymer may independently comprise, on average per alkoxylated nitrogen, at least about 5, or at least about 10, or at least about 15, or at least about 20, alkoxylate groups.

The alkoxylated polyalkyleneimine polymer, preferably alkoxylated PEI, may comprise ethoxylate (EO) groups, propoxylate (PO) groups, or combinations thereof. The alkoxylated polyalkyleneimine polymer, preferably alkoxylated PEI, may comprise ethoxylate (EO) groups. The alkoxylated polyalkyleneimine polymer, preferably alkoxylated PEI, may be free of propoxyate (PO) groups.

The alkoxylated amine, preferably the alkoxylated polyalkyleneimine polymer, more preferably alkoxylated PEI, may comprise on average per alkoxylated nitrogen, about 1-50 ethoxylate (EO) groups and about 0-5 propoxylate (PO) groups. The alkoxylated polyalkyleneimine polymer, preferably alkoxylated PEI, may comprise on average per alkoxylated nitrogen, about 1-50 ethoxylate (EO) groups and is free of propoxylate (PO) groups. The alkoxylated polyalkyleneimine polymer, preferably alkoxylated PEI, may comprise on average per alkoxylated nitrogen, about 10-30 ethoxylate (EO) groups, preferably about 15-25 ethoxylate (EO) groups.

Suitable polyamines include low molecular weight, water soluble, and lightly alkoxylated ethoxylated/propoxylated polyalkyleneamine polymers. By “lightly alkoxylated,” it is meant the polymers average from about 0.5 to about 20, or from 0.5 to about 10, alkoxylations per nitrogen.

The polyamines may be “substantially noncharged,” meaning that there are no more than about 2 positive charges for every about 40 nitrogens present in the backbone of the polyalkyleneamine polymer at pH 10, or at pH 7; it is recognized, however, that the charge density of the polymers may vary with pH.

Suitable alkoxylated polyalkyleneimines, such as PEI600 EO20, are available from BASF (Ludwigshafen, Germany).

Ethylene oxide-propylene oxide-ethylene oxide (EOx₁POyEOx₂) triblock copolymer: In the ethylene oxide-propylene oxide-ethylene oxide (EOx₁POyEOx₂) triblock copolymer, each of x₁ and x₂ is in the range of about 2 to about 140 and y is in the range of from about 15 to about 70. The ethylene oxide-propylene oxide-ethylene oxide (EOx₁POyEOx₂) triblock copolymer preferably has an average propylene oxide chain length of between 20 and 70, preferably between 30 and 60, more preferably between 45 and 55 propylene oxide units.

Preferably, the ethylene oxide-propylene oxide-ethylene oxide (EOx₁POyEOx₂) triblock copolymer has a weight average molecular weight of between about 1000 and about 10,000 Daltons, preferably between about 1500 and about 8000 Daltons, more preferably between about 2000 and about 7000 Daltons, even more preferably between about 2500 and about 5000 Daltons, most preferably between about 3500 and about 3800 Daltons.

Preferably, each ethylene oxide block or chain independently has an average chain length of between 2 and 90, preferably 3 and 50, more preferably between 4 and 20 ethylene oxide units.

Preferably, the copolymer comprises between 10% and 90%, preferably between 15% and 50%, most preferably between 15% and 25% by weight of the copolymer of the combined ethylene-oxide blocks. Most preferably the total ethylene oxide content is equally split over the two ethylene oxide blocks. Equally split herein means each ethylene oxide block comprising on average between 40% and 60% preferably between 45% and 55%, even more preferably between 48% and 52%, most preferably 50% of the total number of ethylene oxide units, the % of both ethylene oxide blocks adding up to 100%. Some ethylene oxide-propylene oxide-ethylene oxide (EOx₁POyEOx₂) triblock copolymer, where each of x₁ and x₂ is in the range of about 2 to about 140 and y is in the range of from about 15 to about 70, improve cleaning.

Preferably the copolymer has a weight average molecular weight between about 3500 and about 3800 Daltons, a propylene oxide content between 45 and 55 propylene oxide units, and an ethylene oxide content of between 4 and 20 ethylene oxide units per ethylene oxide block.

Preferably, the ethylene oxide-propylene oxide-ethylene oxide (EOx₁POyEOx₂) triblock copolymer has a weight average molecular weight of between 1000 and 10,000 Daltons, preferably between 1500 and 8000 Daltons, more preferably between 2000 and 7500 Daltons. Preferably, the copolymer comprises between 10% and 95%, preferably between 12% and 90%, most preferably between 15% and 85% by weight of the copolymer of the combined ethylene-oxide blocks. Some ethylene oxide-propylene oxide-ethylene oxide (EOx₁POyEOx₂) triblock copolymers, where each of x₁ and x₂ is in the range of about 2 to about 140 and y is in the range of from about 15 to about 70, improve dissolution.

Suitable ethylene oxide-propylene oxide-ethylene oxide triblock copolymers are commercially available under the Pluronic PE series from the BASF company, or under the Tergitol L series from the Dow Chemical Company. A particularly suitable material is Pluronic PE 9200.

The solid article of the present disclosure comprises one or more surfactants selected from the group consisting of anionic surfactants, nonionic surfactants, cationic surfactants, zwitterionic surfactants, amphoteric surfactants, polymeric surfactants or combinations thereof. Preferably, selected from the group consisting of anionic surfactants, nonionic surfactants, amphoteric surfactants, and a combination thereof. In a preferred embodiment, the surfactant system comprises linear alkylbenzene sulfonate and alkyl sulfate in a weight ratio of linear alkylbenzene sulfonate to alkyl sulfate of greater than 1, preferably from 40:1 to 20:1. Preferably, the surfactant system further comprises from 0.5 to 10% by weight of the article of C6-C20 linear or branched alkylalkoxylated alcohols (AA) having a weight average degree of alkoxylation ranging from 5 to 15, and combinations thereof.

Alkylalkoxylated Sulfate: The alkylalkoxylated sulfate (AAS) may be an alkylethoxylated sulfate (AES), preferably an ethoxylated C₁₂-C₁₈ alkyl sulfate having an average degree of ethoxylation of from about 0.5 to about 3.0.

Typically, the weight ratio of alkylalkoxylated sulfate to rheology modifier is in the range of from 4:1 to 40:1. The weight ratio of alkylalkoxylated sulfate to rheology modifier may depend on the molecular weight of alcohol precursors of the alkylalkoxylated sulfate, degree of alkoxylation, and blend ratio of LAS/AES in a blended surfactant system. For example, for a degree of ethoxylation of about 1.0 (e.g., NaAE₁S), an NaLAS/NaAE₁S blend ratio of about 1/3, and an AE1 alcohol precursor having a 12-15 carbon chain-length blend, the functional rheology modifier/NaAE1S mass ratio may be at least about 7% to improve dissolution; for a higher MW alcohol precursor having a 14-15 carbon chain-length blend, the preferred functional rheology modifier/NaAE1S mass ratio may be at least about 9%. The level of functional rheology modifier can be adjusted to maintain product dissolution over a range of possible anionic surfactant materials and their blend ratios.

The mass of rheology modifier (RM) relative to mass of NaAES surfactant may follow the following relationship, RM/NaAES≥f(alc)/(a*(LAS/AES)+b), where f(alc) is a function of the structure and molecular weight of the alcohol used to make the AES surfactant, (LAS/AES) is the blend ratio of LAS to AES in the surfactant paste, a~30, and b~2. For a reference blend of predominantly C12-C15 linear alcohol ethoxylate (C25AE1), f(alc)~1.0; for a blend of predominantly C14-C15 linear alcohol ethoxylate (C45AE1), f(alc)~1.2. The above guideline is further dependent on the degree of ethoxylation and any branching structure of ethoxylated alcohol precursors to the AES surfactant. The above guideline can be expressed as a Guidance Ratio, where values of ≥1 may indicate improved dissolution, and values <1 may indicate worse dissolution. The Guidance Ratio is: (RM/NaAES)/(f(alc)/(30*(LAS/AES)+2))

The particle may comprise from about 15 wt % to about 60 wt %, or from 20 wt % to 40 wt % alkylalkoxylated sulfate, or from 30 wt % to 80 wt % or even from 50 wt % to 70 wt % alkylalkoxylated sulfate.

The particle may comprise alkylbenzene sulfonate, for example, linear alkylbenzene sulfonate (LAS). The particle may comprise from 1 wt % to 50 wt % alkylbenzene sulfonate, or from 5 wt % to 30 wt % alkylbenzene sulfonate.

The particle may have a particle size distribution such that the D50 is from greater than about 150 micrometers to less than about 1700 micrometers. The particle may have a particle size distribution such that the D50 is from greater than about 212 micrometers to less than about 1180 micrometers. The particle may have a particle size distribution such that the D50 is from greater than about 300 micrometers to less than about 850 micrometers. The particle may have a particle size distribution such that the D50 is from greater than about 350 micrometers to less than about 700 micrometers. The particle may have a particle size distribution such that the D20 is greater than about 150 micrometers and the D80 is less than about 1400 micrometers. The particle may have a particle size distribution such that the D20 is greater than about 200 micrometers and the D80 is less than about 1180 micrometers. The particle may have a particle size distribution such that the D20 is greater than about 250 micrometers and the D80 is less than about 1000 micrometers. The particle may have a particle size distribution such that the D10 is greater than about 150 micrometers and the D90 is less than about 1400 micrometers. The particle may have a particle size distribution such that the D10 is greater than about 200 micrometers and the D90 is less than about 1180 micrometers. The particle may have a particle size distribution such that the D10 is greater than about 250 micrometers and the D90 is less than about 1000 micrometers.

The particle may be used in a bead-like detergent or derivative thereof. The particle may have a particle size distribution such that the D50 is from greater than about 1 mm to less than about 4.75 mm. The particle may have a particle size distribution such that the D50 is from greater than about 1.7 mm to less than about 3.5 mm. The particle may have a particle size distribution such that the D20 is greater than about 1 mm and the D80 is less than about 4.75 mm. The particle may have a particle size distribution such that the D20 is greater than about 1.7 mm and the D80 is less than about 3.5 mm. The particle may have a particle size distribution such that the D10 is greater than about 1 mm and the D90 is less than about 4.75 mm. The particle may have a particle size distribution such that the D10 is greater than about 1.7 mm and the D90 is less than about 3.5 mm.

The particle's size distribution is measured according to applicants' Granular Size Distribution Test Method.

The particle may comprise from about 10 wt % to about 80 wt % detergent builder, preferably from about 20 wt % to about 60 wt %, preferably from about 30 wt % to about 50 wt %.

The particle may comprise from about 2 wt % to about 40 wt % buffering agent, preferably from about 5 wt % to about 30 wt %, preferably from about 10 wt % to about 20 wt %.

The particle may comprise from about 2 wt % to about 20 wt % chelant, preferably from about 5 wt % to about 10 wt %.

The particle may comprise from about 2 wt % to about 20 wt % dispersant polymer, preferably from about 5 wt % to about 10 wt %.

The particle may comprise from 0.5 wt % to 15 wt % of a soluble film or fiber-structuring polymer. Examples of soluble film or fiber structuring polymers include, but are not limited to, polyvinyl alcohol, polyvinyl pyrollidone, polyethelene oxide, modified starch or cellulose polymers, and mixtures thereof. Such polymers may be present in product recycle streams comprising soluble fiber or film materials, for example unitary dose products comprising pouch material, where it is advantageous to incorporate said recycle materials into the current particle.

The rheology-modified particle may be coated or at least partially coated with a layer composition, for example as disclosed in US2007/0196502. Preferably the layer composition comprises non-surfactant actives. More preferably, said non-surfactant actives are selected from the group consisting builder, buffer and dispersant polymer. Even more preferably, said non-surfactant actives are selected from the group consisting of zeolite-A, sodium carbonate, sodium bicarbonate, and a soluble polycarboxylate polymer. This is especially advantageous when the actives (for non-limiting example AES) are suitable for cleaning in cold-water and/or high hardness wash water conditions. The presence of the actives in the layer promotes the initial dissolution of the cold-water and/or hardness-tolerant chemistry. While not being bound by theory, it is hypothesized that having cold-water and hardness-tolerant chemistries earlier in the order of dissolution can protect the more conventional cleaning actives (for non-limiting example LAS surfactant), resulting in superior overall cleaning performance.

The process of making rheology-modified particles is described in WO 2020/023188 A1, pages 13 to 15.

### pH Adjusting Agent

The single unit dose may comprise one or more Base pH adjusting agents that increase the pH of the wash liquor to a pH greater than 8. Suitable Base pH adjusting agents include, without limitation, compounds that include sulfate ions, dihydrogen phosphate ions, fluoride ions, nitrite ions, acetate ions, hydrogen carbonate ions, hydrogen sulfide ions, ammonia, carbonate ions, hydroxide ions, and combinations thereof. The inclusion of Base pH adjusting agents does not preclude the inclusion of Acid pH adjusting agents such as, for example, citric acid. The single unit dose may include Acid pH adjusting agents provided that the wash liquor final pH is greater than 8, such as for example, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8 or 13.

### Concentrated Surfactant Paste

Concentrated surfactant pastes are intermediate compositions that may be combined with other ingredients to form a rheology modified particle. Concentrated surfactant compositions may comprise, may consist essentially of, or may consist of the following components: a surfactant system that may include an alkylalkoxylated sulfate surfactant; a rheology modifier, as described herein; an organic solvent system; and water. These components are described in more detail below.

The concentrated surfactant composition may comprise: from about 70% to about 90%, by weight of the composition, of a surfactant system, where the surfactant system comprises from about 50%, or from about 60%, or from about 70%, or from about 80%, to about 100%, of alkylalkoxylated sulfate surfactant; from about 0.1% to about 25%, by weight of the composition, of a rheology modifier; less than about 5%, by weight of the composition, of an organic solvent system; and water. The surfactant system of the paste preferably includes LAS co-surfactant. If LAS is included in the surfactant system, the ratio of LAS:AES may be from about 0 to about 1, preferably from about 0.2 to about 0.7, more preferably from about 0.25 to about 0.35.

### Solid Carrier

Suitable solid carriers include inorganic salts, such as sodium carbonate, sodium sulfate and mixtures thereof. Other preferred solid carriers include aluminosilicates, such as zeolite, dried dispersant polymer in a fine powder form, and absorbent grades of fumed or precipitated silica (for example, precipitated hydrophilic silica commercialized by Evonik Industries AG under the trade name SN340). Mixtures of solid carrier materials may also be used.

### Fibrous Structure

Fibrous structures comprise one or more fibrous elements. The fibrous elements can be associated with one another to form a structure. Fibrous structures can include particles within and or on the structure. Fibrous structures can be homogeneous, layered, unitary, zoned, or as otherwise desired, with different active agents defining the various aforesaid portions.

A fibrous structure can comprise one or more layers, the layers together forming a ply.

### Fibrous Elements

The fibrous elements may be water-soluble. The fibrous elements may comprise one or more filament-forming materials and/or one or more active agents, such as a surfactant. The one or more active agents may be releasable from the fibrous element, such as when the fibrous element and/or fibrous structure comprising the fibrous element is exposed to conditions of intended use.

The fibrous elements may be spun from a filament-forming composition, also referred to as fibrous element-forming compositions, via suitable spinning process operations, such as meltblowing, spunbonding, electro-spinning, and/or rotary spinning.

“Filament-forming composition” and/or “fibrous element-forming composition” as used herein means a composition that is suitable for making a fibrous element of the present disclosure such as by meltblowing and/or spunbonding. The filament-forming composition comprises one or more filament-forming materials that exhibit properties that make them suitable for spinning into a fibrous element. The filament-forming material may comprise a polymer. In addition to one or more filament-forming materials, the filament-forming composition may comprise one or more active agents, for example, a surfactant. In addition, the filament-forming composition may comprise one or more polar solvents, such as water, into which one or more, for example all, of the filament-forming materials and/or one or more, for example all, of the active agents are dissolved and/or dispersed prior to spinning a fibrous element, such as a filament from the filament-forming composition.

The filament-forming composition may comprise two or more different filament-forming materials. Thus, the fibrous elements may be monocomponent (one type of filament-forming material) and/or multicomponent, such as bicomponent. The two or more different filament-forming materials may be randomly combined to form a fibrous element. The two or more different filament-forming materials may be orderly combined to form a fibrous element, such as a core and sheath bicomponent fibrous element, which is not considered a random mixture of different filament-forming materials for purposes of the present disclosure. Bicomponent fibrous elements may be in any form, such as side-by-side, core and sheath, islands-in-the-sea and the like.

The fibrous elements may be substantially free of alkylalkoxylated sulfate. Each fibrous element may comprise from about 0%, or from about 0.1%, or from about 5%, or from about 10%, or from about 15%, or from about 20%, or from about 25%, or from about 30%, or from about 35%, or from about 40% to about 0.2%, or to about 1%, or to about 5%, or to about 10%, or to about 15%, or to about 20%, or to about 25%, or to about 30%, or to about 35% or to about 40%, or to about 50% by weight on a dry fibrous element basis of an alkylalkoxylated sulfate. The amount of alkylalkoxylated sulfate in each of the fibrous elements is sufficiently small so as not to affect the processing stability and film dissolution thereof. Alkylalkoxylated sulfates, when dissolved in water, may undergo a highly viscous hexagonal phase at certain concentration ranges, e.g., 30-60% by weight, resulting in a gel-like substance. Therefore, if incorporated into the fibrous elements in a significant amount, alkylalkoxylated sulfates may significantly slow down the dissolution of the water-soluble unit dose articles in water, and worse yet, result in undissolved solids afterwards. Correspondingly, most of such surfactants are formulated into the particles.

The fibrous elements may each contain at least one filament-forming material and an active agent, preferably a surfactant. The surfactant may have a relatively low hydrophilicity, as such a surfactant is less likely to form a viscous, gel-like hexagonal phase when being diluted. By using such a surfactant in forming the filaments, gel-formation during wash may be effectively reduced, which in turn may result in faster dissolution and low or no residues in the wash. The surfactant can be selected, for example, from the group consisting of unalkoxylated C6-C20 linear or branched alkyl sulfates (AS), C6-C20 linear alkylbenzene sulfonates (LAS), and combinations thereof. The surfactant may be a C6-C20 linear alkylbenzene sulfonates (LAS). LAS surfactants are well known in the art and can be readily obtained by sulfonating commercially available linear alkylbenzenes. Exemplary C₆-C₂₀ linear alkylbenzene sulfonates that can be used include alkali metal, alkaline earth metal or ammonium salts of C₆-C₂₀ linear alkylbenzene sulfonic acids, such as the sodium, potassium, magnesium and/or ammonium salts of C₁₁-C₁₈ or C₁₁-C₁₄ linear alkylbenzene sulfonic acids. The sodium or potassium salts of C₁₂ linear alkylbenzene sulfonic acids, for example, the sodium salt of C₁₂ linear alkylbenzene sulfonic acid, i.e., sodium dodecylbenzene sulfonate, may be used as the first surfactant.

The fibrous element may comprise at least about 5%, and/or at least about 10%, and/or at least about 15%, and/or at least about 20%, and/or less than about 80%, and/or less than about 75%, and/or less than about 65%, and/or less than about 60%, and/or less than about 55%, and/or less than about 50%, and/or less than about 45%, and/or less than about 40%, and/or less than about 35%, and/or less than about 30%, and/or less than about 25% by weight on a dry fibrous element basis and/or dry fibrous structure basis of the filament-forming material and greater than about 20%, and/or at least about 35%, and/or at least about 40%, and/or at least about 45%, and/or at least about 50%, and/or at least about 55%, and/or at least about 60%, and/or at least about 65%, and/or at least about 70%, and/or less than about 95%, and/or less than about 90%, and/or less than about 85%, and/or less than about 80%, and/or less than about 75% by weight on a dry fibrous element basis and/or dry fibrous structure basis of an active agent, preferably surfactant. The fibrous element may comprise greater than about 80% by weight on a dry fibrous element basis and/or dry fibrous structure basis of surfactant.

Preferably, each fibrous element may be characterized by a sufficiently high total surfactant content, e.g., at least about 30%, or at least about 40%, or at least about 50%, or at least about 60%, or at least about 70%, by weight on a dry fibrous element basis and/or dry fibrous structure basis of the first surfactant.

The total level of filament-forming materials present in the fibrous element may be from about 5% to less than about 80% by weight on a dry fibrous element basis and/or dry fibrous structure basis and the total level of surfactant present in the fibrous element may be greater than about 20% to about 95% by weight on a dry fibrous element basis and/or dry fibrous structure basis.

One or more of the fibrous elements may comprise at least one additional surfactant selected from the group consisting of other anionic surfactants (i.e., other than AS and LAS), nonionic surfactants, zwitterionic surfactants, amphoteric surfactants, cationic surfactants, and combinations thereof.

A preferred sulphate surfactant is alkyl alkoxylated sulphate, preferably alkyl ethoxylated sulphate (AES), preferably a C8-18 alkyl alkoxylated sulphate, preferably a C8-18 alkyl ethoxylated sulphate, preferably the alkyl alkoxylated sulphate has an average degree of alkoxylation of from 0.5 to 20, preferably from 0.5 to 10, preferably the alkyl alkoxylated sulphate is a C8-18 alkyl ethoxylated sulphate having an average degree of ethoxylation of from 0.5 to 10, preferably from 0.5 to 5, more preferably from 0.5 to 3 or from about 1.5 to 3 or from about 1.8 to 2.5. The alkyl alkoxylated sulfate may have a broad alkoxy distribution or a peaked alkoxy distribution. The alkyl portion of the AES may include, on average, from 13.7 to about 16 or from 13.9 to 14.6 carbons atoms. At least about 50% or at least about 60% of the AES molecule may include having an alkyl portion having 14 or more carbon atoms, preferable from 14 to 18, or from 14 to 17, or from 14 to 16, or from 14 to 15 carbon atoms.

The alkyl sulphate, alkyl alkoxylated sulphate and alkyl benzene sulphonates may be linear or branched, including 2 alkyl substituted or mid chain branched type, substituted or un-substituted, and may be derived from petrochemical material or biomaterial. Preferably, the branching group is an alkyl. Typically, the alkyl is selected from methyl, ethyl, propyl, butyl, pentyl, cyclic alkyl groups and mixtures thereof. Single or multiple alkyl branches could be present on the main hydrocarbyl chain of the starting alcohol(s) used to produce the sulfated anionic surfactant used in the detergent. Most preferably the branched sulfated anionic surfactant is selected from alkyl sulfates, alkyl ethoxy sulfates, and mixtures thereof.

Alkyl sulfates and alkyl alkoxy sulfates are commercially available with a variety of chain lengths, ethoxylation and branching degrees. Commercially available sulfates include those based on Neodol alcohols ex the Shell company, Lial-Isalchem and Safol ex the Sasol company, natural alcohols ex The Procter & Gamble Chemicals company. Suitable surfactants are disclosed in U.S. Pat. Nos. 11,807,829, 9,493,725, and 9,493,726

Other suitable anionic surfactants include C₆-C₂₀ linear or branched alkyl sulfonates, C₆-C₂₀ linear or branched alkyl carboxylates, C₆-C₂₀ linear or branched alkyl phosphates, C₆-C₂₀ linear or branched alkyl phosphonates, C₆-C₂₀ alkyl N-methyl glucose amides, C₆-C₂₀ methyl ester sulfonates (MES), and combinations thereof.

Other suitable anionic surfactants include alkyl ether carboxylates, comprising a C₁₀-C₂₆ linear or branched, preferably C₁₀-C₂₀ linear, most preferably C₁₆-C₁₈ linear alkyl alcohol and from 2 to 20, preferably 7 to 13, more preferably 8 to 12, most preferably 9.5 to 10.5 ethoxylates. The acid form or salt form, such as sodium or ammonium salt, may be used, and the alkyl chain may contain one cis or trans double bond. Alkyl ether carboxylic acids are available from Kao (Akypo®), Huntsman (Empicol®) and Clariant (Emulsogen®).

Other suitable anionic surfactants are rhamnolipids. The rhamnolipids may have a single rhamnose sugar ring or two rhamnose sugar rings.

Suitable nonionic surfactants include alkoxylated fatty alcohols. The nonionic surfactant may be selected from ethoxylated alcohols and ethoxylated alkyl phenols of the formula R(OC₂H₄)ₙOH, wherein R is selected from the group consisting of aliphatic hydrocarbon radicals containing from about 8 to about 15 carbon atoms and alkyl phenyl radicals in which the alkyl groups contain from about 8 to about 12 carbon atoms, and the average value of n is from about 5 to about 15. Non-limiting examples of nonionic surfactants useful herein include: C₈-C₁₈ alkylethoxylates, such as, NEODOL® nonionic surfactants from Shell; C₆-C₁₂ alkyl phenol alkoxylates where the alkoxylate units may be ethyleneoxy units, propyleneoxy units, or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols, BA; C₁₄-C₂₂ mid-chain branched alkylalkoxylates, BAEx, wherein x is from 1 to 30; alkylpolysaccharides; specifically alkylpolyglycosides; polyhydroxy fatty acid amides; and ether capped poly(oxyalkylated) alcohol surfactants. Suitable nonionic detersive surfactants also include alkyl polyglucoside and alkylalkoxylated alcohol. Suitable nonionic surfactants also include those sold under the tradename Lutensol® from BASF. The alkoxylated fatty alcohol may have a broad alkoxy distribution for example Alfonic 1214-9 Ethoxylate or a peaked alkoxy distribution for example Novel 1214-9 both commercially available from Sasol. Suitable surfactants are disclosed in U.S. Pat. No. 11,795,131

Suitable examples of zwitterionic surfactants include: derivatives of secondary and tertiary amines, including derivatives of heterocyclic secondary and tertiary amines; derivatives of quaternary ammonium, quaternary phosphonium or tertiary sulfonium compounds; betaines, including alkyl dimethyl betaine, cocodimethyl amidopropyl betaine, and sulfo and hydroxy betaines; C₈ to C₁₈ (e.g., from C₁₂ to C₁₈) amine oxides; N-alkyl-N,N-dimethylammino-1-propane sulfonate, where the alkyl group can be C₈ to C₁₈.

Suitable amphoteric surfactants include aliphatic derivatives of secondary or tertiary amines, or aliphatic derivatives of heterocyclic secondary and tertiary amines in which the aliphatic radical may be straight or branched-chain and where one of the aliphatic substituents contains at least about 8 carbon atoms, or from about 8 to about 18 carbon atoms, and at least one of the aliphatic substituents contains an anionic water-solubilizing group, e.g. carboxy, sulfonate, sulfate. Suitable amphoteric surfactants also include sarcosinates, glycinates, taurinates, and mixtures thereof.

Suitable cationic surfactants include alkyl pyridinium compounds, alkyl quaternary ammonium compounds, alkyl quaternary phosphonium compounds, alkyl ternary sulphonium compounds, and mixtures thereof.

Preferred cationic surfactants are quaternary ammonium compounds having the general formula:

(R)(R1)(R2)(R3)N+X−
wherein, R is a linear or branched, substituted or unsubstituted C6-18 alkyl or alkenyl moiety, R1 and R2 are independently selected from methyl or ethyl moieties, R3 is a hydroxyl, hydroxymethyl or a hydroxyethyl moiety, X is an anion which provides charge neutrality, preferred anions include: halides, preferably chloride; sulphate; and sulphonate.

The fibrous elements may comprise a surfactant system containing only anionic surfactants, e.g., either a single anionic surfactant or a combination of two or more different anionic surfactants. Alternatively, the fibrous elements may include a composite surfactant system, e.g., containing a combination of one or more anionic surfactants with one or more nonionic surfactants, or a combination of one or more anionic surfactants with one or more zwitterionic surfactants, or a combination of one or more anionic surfactants with one or more amphoteric surfactants, or a combination of one or more anionic surfactants with one or more cationic surfactants, or a combination of all the above-mentioned types of surfactants (i.e., anionic, nonionic and amphoteric).

In general, fibrous elements are elongated particulates having a length greatly exceeding average diameter, e.g., a length to average diameter ratio of at least about 10. A fibrous element may be a filament or a fiber. Filaments are relatively longer than fibers. A filament may have a length of greater than or equal to about 5.08 cm (2 in.), and/or greater than or equal to about 7.62 cm (3 in.), and/or greater than or equal to about 10.16 cm (4 in.), and/or greater than or equal to about 15.24 cm (6 in.). A fiber may have a length of less than about 5.08 cm (2 in.), and/or less than about 3.81 cm (1.5 in.), and/or less than about 2.54 cm (1 in.).

The one or more filament-forming materials and active agents may be present in the fibrous element at a weight ratio of total level of filament-forming materials to active agents of about 2.0 or less, and/or about 1.85 or less, and/or less than about 1.7, and/or less than about 1.6, and/or less than about 1.5, and/or less than about 1.3, and/or less than about 1.2, and/or less than about 1, and/or less than about 0.7, and/or less than about 0.5, and/or less than about 0.4, and/or less than about 0.3, and/or greater than about 0.1, and/or greater than about 0.15, and/or greater than about 0.2. The one or more filament-forming materials and active agents may be present in the fibrous element at a weight ratio of total level of filament-forming materials to active agents of about 0.2 to about 0.7.

The fibrous element may comprise from about 10% to less than about 80% by weight on a dry fibrous element basis and/or dry fibrous structure basis of a filament-forming material, such as polyvinyl alcohol polymer, starch polymer, and/or carboxymethylcellulose polymer, and greater than about 20% to about 90% by weight on a dry fibrous element basis and/or dry fibrous structure basis of an active agent, such as surfactant. The fibrous element may further comprise a plasticizer, such as glycerin, and/or additional pH adjusting agents, such as citric acid. The fibrous element may have a weight ratio of filament-forming material to active agent of about 2.0 or less. The filament-forming material may be selected from the group consisting of polyvinyl alcohol, starch, carboxymethylcellulose, polyethylene oxide, and other suitable polymers, especially hydroxyl-containing polymers and their derivatives. The filament-forming material may range in weight average molecular weight from about 100,000 g/mol to about 3,000,000 g/mol. It is believed that in this range, the filament-forming material may provide extensional rheology, without being so elastic that fiber attenuation is inhibited in the fiber-making process.

The one or more active agents may be releasable and/or released when the fibrous element and/or fibrous structure comprising the fibrous element is exposed to conditions of intended use. The one or more active agents in the fibrous element may be selected from the group consisting of surfactants, organic polymeric compounds, and mixtures thereof.

The fibrous elements may exhibit a diameter of less than about 300 μm, and/or less than about 75 μm, and/or less than about 50 μm, and/or less than about 25 μm, and/or less than about 10 μm, and/or less than about 5 μm, and/or less than about 1 μm as measured according to the Diameter Test Method described herein. The fibrous elements may exhibit a diameter of greater than about 1 μm as measured according to the Diameter Test Method described herein. The diameter of a fibrous element may be used to control the rate of release of one or more active agents present in the fibrous element and/or the rate of loss and/or altering of the fibrous element's physical structure.

The fibrous element may comprise two or more different active agents, which are compatible or incompatible with one another. The fibrous element may comprise an active agent within the fibrous element and an active agent on an external surface of the fibrous element, such as an active agent coating on the fibrous element. The active agent on the external surface of the fibrous element may be the same or different from the active agent present in the fibrous element.

If different, the active agents may be compatible or incompatible with one another. The one or more active agents may be uniformly distributed or substantially uniformly distributed throughout the fibrous element. The one or more active agents may be distributed as discrete regions within the fibrous element.

### Active Agents

The water-soluble unit dose articles described herein comprise a surfactant system, a serine protease and a metalloprotease. The article may further comprise one or more active agents. The active agents may be present in the fibrous elements (as described above), in the particles (as described above), or as a premix in the article. Premixes for example, may be slurries of active agents that are combined with aqueous absorbents. The active agent may be selected from the group consisting of a surfactant, a structurant, a builder, an organic polymeric compound, an enzyme, an enzyme stabilizer, a bleach system, a brightener, a hueing agent, a chelating agent, a suds suppressor, a conditioning agent, a humectant, a perfume, a perfume microcapsule, a filler or carrier, an alkalinity system, a pH control system, a buffer, an alkanolamine, and mixtures thereof.

### Surfactant

The surfactant may be selected from the group consisting of anionic surfactants, nonionic surfactants, cationic surfactants, zwitterionic surfactants, amphoteric surfactants, ampholytic surfactants, and mixtures thereof. These surfactants are described in more detail above.

### Enzymes

### Serine Proteases

Suitable proteases for use in combination with metalloproteases are serine proteases, including neutral or alkaline microbial serine proteases, such as subtilisins (EC 3.4.21.62). Suitable proteases include those of animal, vegetable or microbial origin. In one aspect, such suitable protease may be of microbial origin. The suitable proteases include chemically or genetically modified mutants of the aforementioned suitable proteases. In one aspect, the suitable protease may be a serine protease, such as an alkaline microbial protease or/and a trypsin-type protease. Examples of suitable neutral or alkaline proteases include:
i) subtilisins (EC 3.4.21.62), especially those derived from Bacillus, such as Bacillus sp., B. lentus, B. alkalophilus, B. subtilis, B. amyloliquefaciens, B. gibsonii, B. akibaii, Bacillus Clausii and B. clarkii described in WO2004067737, WO2015091989, WO2015091990, WO2015024739, WO2015143360, U.S. Pat. No. 6,312,936 B1, U.S. Pat. Nos. 5,679,630, 4,760,025, WO03/055974, WO03/054185, WO03/054184, WO2017/215925, DE102006022216A1, WO2015089447, WO2015089441, WO2016066756, WO2016066757, WO2016069557, WO2016069563, WO2016069569, WO2016174234, WO2017/089093, WO2020/156419, WO2016/183509. Specifically, mutations S9R, A15T, V66A, A188P, V199I, N212D, Q239R, N255D, X9E, X200L, X256E, X9R, X19L, X60D (Savinase numbering system)
ii) subtilisins from B. Pumilus such as the ones described in DE102006022224A1, WO2020/221578, WO2020/221579, WO2020/221580, including variants comprising amino acid substitutions in at least one or more of the positions selected from 9, 130, 133, 144, 224, 252, 271 (BPN′ numbering system).
iii) trypsin-type or chymotrypsin-type proteases, such as trypsin (e.g., of porcine or bovine origin), including the Fusarium protease described in WO 89/06270 and the chymotrypsin
iv) proteases derived from Cellumonas described in WO 05/052161 and WO 05/052146
v) proteases having at least 90% identity to the subtilase from Bacillus sp. TY145, NCIMB 40339, described in WO92/17577 (Novozymes A/S), including the variants of this Bacillus sp TY145 subtilase described in WO2015024739, and WO2016066757.

The most preferred serine protease for use herein is a variant, wherein said variant has at least 90%, preferably at least 95% amino acid identity with Bacillus lentus subtilisin GG36 protease comprising the amino acid sequence shown in SEQ ID NO:2 and said variant comprises an amino acid sequence comprising one of the combinations of amino acid substitutions: T022A-S103A-V104IN116L-G159D-S188D-A232V-Q245R-N248D-E271F, T022A-S101G-V104I-N116L-G159D-S188D-A232V-Q245R-N248D-E271F, T022A-S101G-S103A-V104I-N116L-S188D-A232V-Q245R-N248D-E271F, T022A-S101G-S103A-V104I-N116L-G159D-S188D-Q245R-N248D-E271F, T022A-S101G-S103A-V104I-N116L-G159D-S188D-A232V-N248D-E271F, T022A-S101G-S103A-V104I-N116L-G159D-S188D-A232V-Q245R-E271F, T022A-S101G-S103A-V104I-N116L-G159D-S188D-A232V-Q245R-N248D-E271F, T022A-T033S-S101G-S103A-V104I-N116L-G159D-S188D-T213A-A232V-Q245R-N248D-E271F, T022A-V068A-S101G-S103A-V104IN116L-G159D-S188D-T213A-A232V-Q245R-N248D-E271F, T022A-S101G-S103A-V104IN116L-G159D-S188D-T213A-A232V-Q245R-N248D-E271F, T022A-S101G-S103A-V104IN116L-S128L-G159D-S188D-A232V-Q245R-N248D-E271F, T022A-V068A-S101G-S103A-V104I-N116L-G159D-S188D-A232V-Q245R-N248D-E271F, T022A-S101G-S103A-V104I-N116L-G159D-S188D-A232V-Q245R-N248D, T022A-S101G-S103A-V104I-N116L-S128L-G159D-S188D-T213A-A232V-Q245R-N248D-E271F, T022A-N062D-S101G-S103A-V104I-N116L-G159D-S188D-T213A-A232V-Q245R-N248D-E271F, T022A-T033S-S101G-S103A-V104I-N116L-S128L-G159D-S188D-A232V-Q245R-N248D-E271F, T022A-T033S-S101G-S103A-V104I-N116L-G159D-S188D-A232V-Q245R-N248D-E271F, T022A-T033S-S101G-S103A-V104I-N116L-S128L-G159D-S188D-T213A-A232V-Q245R-N248D-E271F, wherein amino acid positions of the subtilisin variant are numbered according to the numbering of corresponding amino acid positions in the amino acid sequence of Bacillus amyloliquefaciens subtilisin BPN′ shown in SEQ ID NO:3, preferably said variant comprising a combination of amino acid substitutions selected from T022A-S103A-V104I-N116L-G159D-S188D-A232V-Q245R-N248D-E271F, T022A-S101G-V104I-N116L-G159D-S188D-A232V-Q245R-N248D-E271F, T022A-S101G-S103A-V104I-N116L-S188D-A232V-Q245R-N248D-E271F, T022A-S101G-S103A-V104I-N116L-G159D-S188D-Q245R-N248D-E271F, T022A-S101G-S103A-V104I-N116L-G159D-S188D-A232V-N248D-E271F, T022A-S101G-S103A-V104I-N116L-G159D-S188D-A232V-Q245R-E271F, T022A-S101G-S103A-V104I-N116L-G159D-S188D-A232V-Q245R-N248D-E271F, T022A-T033S-S101G-S103A-V104I-N116L-G159D-S188D-T213A-A232V-Q245R-N248D-E271F, T022A-V068A-S101G-S103A-V104I-N116L-G159D-S188D-T213A-A232V-Q245R-N248D-E271F, T022A-S101G-S103A-V104I-N116L-G159D-S188D-T213A-A232V-Q245R-N248D-E271F, T022A-S101G-S103A-V104I-N116L-S128L-G159D-S188D-A232V-Q245R-N248D-E271F, T022A-V068A-S101G-S103A-V104I-N116L-G159D-S188D-A232V-Q245R-N248D-E271F, and wherein amino acid positions of the subtilisin variant are numbered according to the numbering of corresponding amino acid positions in the amino acid sequence of Bacillus amyloliquefaciens subtilisin BPN′ shown in SEQ ID NO:3.

Especially preferred said variant has at least 90%, preferably at least 95% amino acid identity with Bacillus lentus subtilisin GG36 protease comprising the amino acid sequence shown in SEQ ID NO:2 and said variant comprises an amino acid sequence comprising one of the combinations of amino acid substitutions: T022A-S101G-S103A-V104I-N116L-G159D-S188D-A232V-Q245R-N248D-E271F, T022A-S101G-S103A-V104I-N116L-G159D-S188D-A232V-Q245R-N248D-E271F and T22R+S101G+S103A+V104I+A232V+Q245R and wherein amino acid positions of the subtilisin variant are numbered according to the numbering of corresponding amino acid positions in the amino acid sequence of Bacillus amyloliquefaciens subtilisin BPN′ shown in SEQ ID NO:3.

Other proteases suitable for the detergent of the present disclosure are polypeptides demonstrating at least 90%, preferably at least 95%, more preferably at least 98%, even more preferably at least 99% and especially 100% identity with the wild-type enzyme from Bacillus lentus, comprising mutations in one or more, preferably two or more and more preferably three or more of the following positions, using the BPN′ numbering system and amino acid abbreviations as illustrated in WO00/37627, which is incorporated herein by reference: S9R, A15T, V68A, N76D, N87S, S99D, S99SD, S99A, S101G, S101M, S103A, V104N/I, G118V, G118R, S128L, P129Q, S130A, Y167A, R170S, A194P, V205I, Q206L/D/E, Y209W, M222S, Q245R and/or M222S.

Other suitable protease is selected from the group of proteases comprising the below mutations (BPN′ numbering system) versus either the PB92 wild-type (SEQ ID NO:2 in WO 08/010925) or the subtilisin 309 wild-type (sequence as per PB92 backbone, except comprising a natural variation of N87S).
(i) G118V+S128L+P129Q+S130A
(ii) S101M+G118V+S128L+P129Q+S130A
(iii) N76D+N87R+G118R+S128L+P129Q+S130A+S188D+N248R
(iv) N76D+N87R+G118R+S128L+P129Q+S130A+S188D+V244R
(v) N76D+N87R+G118R+S128L+P129Q+S130A
(vi) V68A+N87S+S101G+V104N
(vii) S99AD
(viii) S9R+A15T+V68A+N218D+Q245R

Other suitable protease is a subtilase variant derived from B. amyloliquefaciens (BPN′), as described in WO2011/072117. Especially useful BPN′ variant comprises mutation in one or more of the following positions: X003Q, X006W, X022Y, X024K, X024Q, X024G, X033T, X045V, X053G, X055P, S063T, X076D, X078N, X087D, X101N, X109Q, X118R, X128A, X128S, X145R, X166Q, X169A, X162Q, X182Q, X183N, S183T, X204Q, X206Y, X217Q, Y217L, X218S, X222Q, X248A or X254A; (in BPN′ numbering system).

Suitable commercially available additional protease enzymes include:
i) those sold under the trade names Alcalase®, Savinase®, Primase®, Durazym®, Polarzyme®, Kannase®, Liquanase®, Liquanase Ultra®, Savinase Ultra®, Liquanase® Evity®, Savinase® Evity®, Ovozyme®, Neutrase®, Everlase®, Coronase®, Blaze®, Blaze Ultra®, Blaze® Evity®, Blaze® Exceed, Blaze® Pro, Esperase®, Progress® Uno, Progress® Excel, Progress® Key, Ronozyme®, Vinzon® and Het Ultra® by Novozymes A/S (Denmark);
those sold under the tradename Maxatase®, Maxacal®, Maxapem®, Properase®, Purafect®, Purafect Prime®, Purafect Ox®, FN3®, FN4®, Excellase®, Ultimase® and Purafect OXP®, Preferenz® by IFF (formerly Dupont);
ii) those sold under the tradename Opticlean® and Optimase® by Solvay Enzymes;
those available from Henkel/Kemira, namely BLAP (sequence shown in FIG. 29 of U.S. Pat. No. 5,352,604 with the following mutations S99D+S101R+S103A+V104I+G159S, hereinafter referred to as BLAP), BLAP R (BLAP with S3T+V4I+V199M+V205I+L217D), BLAP X (BLAP with S3T+V4I+V205I) and BLAP F49 (BLAP with S3T+V4I+A194P+V199M+V205I+L217D); and can comprise a further mutation 101E/D, S156D, L262E, 206A/L/S/T/, 209K/V/W, 215W, 216N/S/T; and
iii) those sold under the tradename and Lavergy®, Lavergy® Pro, Lavergy® C Bright from BASF.

Especially preferred for use herein in combination with the metalloprotease are commercial proteases selected from the group consisting of Preferenz®, Properase®, Blaze®, Ultimase®, Everlase®, Savinase®, Excellase®, Blaze Ultra®, BLAP and BLAP variants (Lavergy® Pro).

Preferred levels of protease in the product of the present disclosure include from about 0.05 to about 10 mg, more preferably from about 0.5 to about 7 mg and especially from about 1 to about 6 mg of active protease/g of the article.

### Metalloprotease

The term “metalloprotease” is used herein in its conventional sense within the field of enzyme research and denotes a protease that requires a bound metal ion in order to be catalytically active. This is usually a zinc ion. The metalloproteases are preferably neutral metalloproteases, i.e. those that inter alia are active even at a neutral pH and require zinc ions for their catalytic effect. The molecular weight of proteases of this kind is usually in the range of from about 30 to about 40 kDa. The neutral metalloproteases are also referred “neutral metalloendopeptidases”.

The metalloprotease of the water-soluble unit-dose solid detergent article is a metalloprotease variant. The variant has a sequence identity of at least 60%, preferably at least 80%, more preferably at least 90%, more preferably at least 95%, more preferably at least 98% to the metalloprotease of SEQ ID NO:1 (SEQ ID NO:33 of WO2014/194034).

The water-soluble unit-dose solid detergent article can comprise additional enzymes.

Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is an enzyme cocktail that may comprise, for example, a protease and one or more non-protease enzymes such as, for example, a lipase in conjunction with amylase or any of those listed above.

When present in a detergent composition, the aforementioned additional enzymes may be present at levels from about 0.00001% to about 2%, from about 0.0001% to about 1% or even from about 0.001% to about 0.5% enzyme protein by weight of the composition. The compositions disclosed herein may comprise from about 0.001% to about 1% by weight of an enzyme (as an adjunct), which may be selected from the group consisting of lipase, amylase, protease, mannanase, cellulase, pectinase, and mixtures thereof.

### Builders

Suitable builders include aluminosilicates (e.g., zeolite builders, such as zeolite A, zeolite P, and zeolite MAP), silicates, phosphates, such as polyphosphates (e.g., sodium tri-polyphosphate), especially sodium salts thereof; carbonates, bicarbonates, sesquicarbonates, and carbonate minerals other than sodium carbonate or sesquicarbonate; organic mono-, di-, tri-, and tetracarboxylates, especially water-soluble nonsurfactant carboxylates in acid, sodium, potassium or alkanolammonium salt form, as well as oligomeric or water-soluble low molecular weight polymer carboxylates including aliphatic and aromatic types; and phytic acid. Additional suitable builders may be selected from citric acid, lactic acid, fatty acid, polycarboxylate builders, for example, copolymers of acrylic acid, copolymers of acrylic acid and maleic acid, and copolymers of acrylic acid and/or maleic acid, and other suitable ethylenic monomers with various types of additional functionalities. Alternatively, the composition may be substantially free of builder.

### Polymeric Dispersing Agents

Suitable polymers include, but are not limited to, polymeric carboxylates, such as polyacrylates, poly acrylic-maleic co-polymers, and sulfonated modifications thereof, for example, a hydrophobically modified sulfonated acrylic acid copolymer. The polymer may be a cellulosic based polymer, a polyester, a polyterephthalate, a polyethylene glycol, an ethylene oxide-propylene oxide-ethylene oxide (EOx₁POyEOx₂) triblock copolymer, where each of x₁ and x₂ is in the range of about 2 to about 140 and y is in the range of from about 15 to about 70, a polyethyleneimine, any modified variant thereof, such as polyethylene glycol having grafted vinyl and/or alcohol moieties, and any combination thereof. In some cases, the dispersant polymer may also function as a rheology modifier, as described above.

Suitable graft polymers include polyalkylene oxide backbone (A) as a graft base and polymeric sidechains (B) grafted thereon. The polymeric sidechains (B) are obtainable by polymerization of at least one vinyl ester monomer. The polyalkylene oxide backbone (A) is obtainable by polymerization of at least one monomers selected from the group of ethylene oxide, 1,2-propylene oxide, 1,2-butylene oxide, 2,3-butylene oxide, 1,2-pentene oxide or 2,3-pentene oxide. Such graft polymers are known as effective soil suspension polymers for hydrophobic and hydrophilic stains, surfactant boosters, and sometimes as dye transfer inhibitors.

Suitable graft polymers include amphilic graft co-polymer comprises polyethylene glycol backbone (A) as a graft base, and at least one pendant sidechains (B) selected from polyvinyl acetate, polyvinyl alcohol and mixtures thereof. A preferred graft polymer of this type is Sokalan HP22 available from BASF.

Suitable graft polymers are also described in WO2007/138053 as amphiphilic graft polymers based on water-soluble polyalkylene oxides (A) as a graft base and side chains formed by polymerization of a vinyl ester component (B), said polymers having an average of <one graft site per 50 alkylene oxide units and mean molar masses M of from 3 000 to 100 000. One specific preferred graft polymer of this type is polyvinyl acetate grafted polyethylene oxide copolymer having a polyethylene oxide as graft base and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units. The most preferred polymer of this type is available from BASF as Sokalan PG101.

Suitable graft polymer also include graft polymer comprising a block copolymer backbone (A) as a graft base, wherein said block copolymer backbone (A) is obtainable by polymerization of at least two monomers selected from the group of ethylene oxide, 1,2-propylene oxide, 1,2-butylene oxide, 2,3-butylene oxide, 1,2-pentene oxide or 2,3-pentene oxide, wherein the number (x) of individual blocks within the block copolymer backbone (A) is an integer, wherein x is from 2 to 10 and preferably 3 to 5, and (B) polymeric sidechains grafted onto the block copolymer backbone, wherein said polymeric sidechains (B) are obtainable by polymerization of at least one vinyl ester monomer. Suitable graft polymers of this type are described in WO2021/160795 and WO2021/160851, these polymers have improved biodegradation profiles.

Suitable graft polymer also include graft polymer comprising a polyalkylene oxide backbone (A) which has a number average molecular weight of from about 1000 to about 20,000 Daltons and is based on ethylene oxide, propylene oxide, or butylene oxide; and side chains derived from N-vinylpyrrolidone (B), and side chains derived from vinyl ester (C) derived from a saturated monocarboxylic acid containing from 1 to 6 carbon atoms and/or a methyl or ethyl ester of acrylic or methacrylic acid. Such graft polymers are described in WO2020005476 and can be used as dye transfer inhibitors.

Suitable polyethyleneimine polymers include propoxylated polyalkylenimine (e.g., PEI) polymers. The propoxylated polyalkylenimine (e.g., PEI) polymers may also be ethoxylated. The propoxylated polyalkylenimine (e.g., PEI) polymers may have inner polyethylene oxide blocks and outer polypropylene oxide blocks, the degree of ethoxylation and the degree of propoxylation not going above or below specific limiting values. The ratio of polyethylene blocks to polypropylene blocks (n/p) may be from about 0.6, or from about 0.8, or from about 1, to a maximum of about 10, or a maximum of about 5, or a maximum of about 3. The n/p ratio may be about 2. The propoxylated polyalkylenimines may have PEI backbones having weight average molecular weights (as determined prior to alkoxylation) of from about 200 g/mol to about 1200 g/mol, or from about 400 g/mol to about 800 g/mol, or about 600 g/mol. The molecular weight of the propoxylated polyalkylenimines may be from about 8,000 to about 20,000 g/mol, or from about 10,000 to about 15,000 g/mol, or about 12,000 g/mol.

Suitable propoxylated polyalkylenimine polymers may include compounds of the following structure:

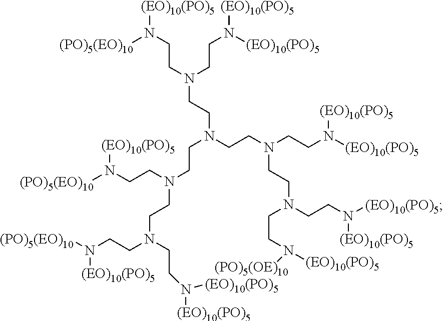

where EOs are ethoxylate groups and POs are propoxylate groups. The compound shown above is a PEI where the molar ratio of EO:PO is 10:5 (e.g., 2:1). Other similar, suitable compounds may include EO and PO groups present in a molar ratio of about 10:5 or about 24:16.

### Soil Release Polymer

Suitable soil release polymers have a structure as defined by one of the following structures (I), (II) or (III):

—[(OCHR¹—CHR²)ₐ—O—OC—Ar—CO—]_{d} (I)

—[(OCHR³—CHR⁴)_{b}—O—OC—sAr—CO—]ₑ (II)

—[(OCHR⁵—CHR⁶)_{c}—OR⁷]_{f} (III)
wherein:
a, b and c are from 1 to 200;
d, e and f are from 1 to 50;
Ar is a 1,4-substituted phenylene;
sAr is 1,3-substituted phenylene substituted in position 5 with SO₃Me;
Me is Na, Li, K, Mg/2, Ca/2, Al/3, ammonium, mono-, di-, tri-, or tetraalkylammonium wherein the alkyl groups are C₁-C₁₈ alkyl or C₂-C₁₀ hydroxyalkyl, or mixtures thereof;
R¹, R², R³, R⁴, R⁵ and R⁶ are independently selected from H or C₁-C₁₈ n- or iso-alkyl; and
R⁷ is a linear or branched C₁-C₁₈ alkyl, or a linear or branched C₂-C₃₀ alkenyl, or a cycloalkyl group with 5 to 9 carbon atoms, or a C₈-C₃₀ aryl group, or a C₆-C₃₀ arylalkyl group.

Suitable soil release polymers are polyester soil release polymers such as Repel-o-tex polymers, including Repel-o-tex SF, SF-2 and SRP6 supplied by Rhodia. Other suitable soil release polymers include Texcare polymers, including Texcare SRA100, SRA300, SRN100, SRN170, SRN240, SRN300 and SRN325 supplied by Clariant. Other suitable soil release polymers are Marloquest polymers, such as Marloquest SL supplied by Sasol.

### Cellulosic Polymer

Suitable cellulosic polymers including those selected from alkyl cellulose, alkyl alkoxyalkyl cellulose, carboxyalkyl cellulose, alkyl carboxyalkyl cellulose. The cellulosic polymers may be selected from the group consisting of carboxymethyl cellulose, methyl cellulose, methyl hydroxyethyl cellulose, methyl carboxymethyl cellulose, and mixtures thereof. In one aspect, the carboxymethyl cellulose has a degree of carboxymethyl substitution from 0.5 to 0.9 and a molecular weight from 100,000 Da to 300,000 Da.

### Amines

Non-limiting examples of amines may include, but are not limited to, polyetheramines, polyamines, oligoamines, triamines, diamines, pentamines, tetraamines, or combinations thereof. Specific examples of suitable additional amines include tetraethylenepentamine, triethylenetetraamine, diethylenetriamine, or a mixture thereof.

### Bleaching Agents

Suitable bleaching agents other than bleaching catalysts include photobleaches, bleach activators, hydrogen peroxide, sources of hydrogen peroxide, pre-formed peracids and mixtures thereof. In general, when a bleaching agent is used, the detergent compositions of the present disclosure may comprise from about 0.1% to about 50% or even from about 0.1% to about 25% bleaching agent by weight of the detergent composition.

### Bleach Catalysts

Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof.

### Brighteners

Commercial fluorescent brighteners suitable for the present disclosure can be classified into subgroups, including but not limited to: derivatives of stilbene, pyrazoline, coumarin, benzoxazoles, carboxylic acid, methinecyanines, dibenzothiophene-5,5-dioxide, azoles, 5- and 6-membered-ring heterocycles, and other miscellaneous agents.

The fluorescent brightener may be selected from the group consisting of disodium 4,4′-bis{[4-anilino-6-morpholino-s-triazin-2-yl]-amino}-2,2′-stilbenedisulfonate (brightener 15, commercially available under the tradename Tinopal AMS-GX by BASF), disodium 4,4′-bis{[4-anilino-6-(N-2-bis-hydroxyethyl)-s-triazine-2-yl]-amino}-2,2′-stilbenedisulonate (commercially available under the tradename Tinopal UNPA-GX by BASF), disodium 4,4′-bis{[4-anilino-6-(N-2-hydroxyethyl-N-methylamino)-s-triazine-2-yl]-amino}-2,2′-stilbenedisulfonate (commercially available under the tradename Tinopal 5BM-GX by BASF). More preferably, the fluorescent brightener is disodium 4,4′-bis{[4-anilino-6-morpholino-s-triazin-2-yl]-amino}-2,2′-stilbenedisulfonate.

The brighteners may be added in particulate form or as a premix with a suitable solvent, for example nonionic surfactant, propanediol.

### Fabric Hueing Agents

A fabric hueing agent (sometimes referred to as shading, bluing or whitening agents) typically provides a blue or violet shade to fabric. Hueing agents can be used either alone or in combination to create a specific shade of hueing and/or to shade different fabric types. This may be provided for example by mixing a red and green-blue dye to yield a blue or violet shade. Hueing agents may be selected from any known chemical class of dye, including but not limited to acridine, anthraquinone (including polycyclic quinones), azine, azo (e.g., monoazo, disazo, trisazo, tetrakisazo, polyazo), including premetallized azo, benzodifurane and benzodifuranone, carotenoid, coumarin, cyanine, diazahemicyanine, diphenylmethane, formazan, hemicyanine, indigoids, methane, naphthalimides, naphthoquinone, nitro and nitroso, oxazine, phthalocyanine, pyrazoles, stilbene, styryl, triarylmethane, triphenylmethane, xanthenes and mixtures thereof.

Suitable fabric hueing agents include dyes, dye-clay conjugates, and organic and inorganic pigments. Suitable dyes also include small molecule dyes and polymeric dyes. Suitable small molecule dyes include small molecule dyes selected from the group consisting of dyes falling into the Color Index (C.I.) classifications of Direct, Basic, Reactive or hydrolysed Reactive, Solvent or Disperse dyes for example that are classified as Blue, Violet, Red, Green or Black, and provide the desired shade either alone or in combination. Suitable polymeric dyes include polymeric dyes selected from the group consisting of polymers containing covalently bound (sometimes referred to as conjugated) chromogens, (dye-polymer conjugates), for example polymers with chromogens co-polymerized into the backbone of the polymer and mixtures thereof. Suitable polymeric dyes also include polymeric dyes selected from the group consisting of fabric-substantive colorants sold under the name of Liquitint® (Milliken, Spartanburg, South Carolina, USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. Suitable polymeric dyes also include polymeric dyes selected from the group consisting of Liquitint® Violet CT, carboxymethyl cellulose (CMC) covalently bound to a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colourants, alkoxylated thiophene polymeric colourants, and mixtures thereof.

The aforementioned fabric hueing agents can be used in combination (any mixture of fabric hueing agents can be used).

### Encapsulates

An encapsulate may comprise a core, a shell having an inner and outer surface, said shell encapsulating said core. The core may comprise any laundry care adjunct, though typically the core may comprise material selected from the group consisting of perfumes; brighteners; hueing dyes; insect repellants; silicones; waxes; flavors; vitamins; fabric softening agents; skin care agents in one aspect, paraffins; enzymes; anti-bacterial agents; bleaches; sensates; and mixtures thereof; and said shell may comprise a material selected from the group consisting of polyethylenes; polyamides; polyvinylalcohols, optionally containing other co-monomers; polystyrenes; polyisoprenes; polycarbonates; polyesters; polyacrylates; aminoplasts, in one aspect said aminoplast may comprise a polyureas, polyurethane, and/or polyureaurethane, in one aspect said polyurea may comprise polyoxymethyleneurea and/or melamine formaldehyde; polyolefins; polysaccharides, in one aspect said polysaccharide may comprise alginate and/or chitosan; gelatin; shellac; epoxy resins; vinyl polymers; water insoluble inorganics; silicone; and mixtures thereof.

Preferred encapsulates comprise perfume. Preferred encapsulates comprise a shell which may comprise melamine formaldehyde and/or cross linked melamine formaldehyde. Other preferred capsules comprise a polyacrylate based shell. Preferred encapsulates comprise a core material and a shell, said shell at least partially surrounding said core material, is disclosed. At least 75%, 85% or even 90% of said encapsulates may have a fracture strength of from 0.2 MPa to 10 MPa, and a benefit agent leakage of from 0% to 20%, or even less than 10% or 5% based on total initial encapsulated benefit agent. Preferred are those in which at least 75%, 85% or even 90% of said encapsulates may have (i) a particle size of from 1 microns to 80 microns, 5 microns to 60 microns, from 10 microns to 50 microns, or even from 15 microns to 40 microns, and/or (ii) at least 75%, 85% or even 90% of said encapsulates may have a particle wall thickness of from 30 nm to 250 nm, from 80 nm to 180 nm, or even from 100 nm to 160 nm. Formaldehyde scavengers may be employed with the encapsulates, for example, in a capsule slurry and/or added to a composition before, during or after the encapsulates are added to such composition.

Suitable capsules that can be made using known processes. Alternatively, suitable capsules can be purchased from Encapsys LLC of Appleton, Wisconsin USA. In a preferred aspect the composition may comprise a deposition aid, preferably in addition to encapsulates. Preferred deposition aids are selected from the group consisting of cationic and nonionic polymers. Suitable polymers include cationic starches, cationic hydroxyethylcellulose, polyvinylformaldehyde, locust bean gum, mannans, xyloglucans, tamarind gum, polyethyleneterephthalate and polymers containing dimethylaminoethyl methacrylate, optionally with one or more monomers selected from the group comprising acrylic acid and acrylamide.

### Perfumes

Non-limiting examples of perfume and perfumery ingredients include, but are not limited to, aldehydes, ketones, esters, and the like. Other examples include various natural extracts and essences which can comprise complex mixtures of ingredients, such as orange oil, lemon oil, rose extract, lavender, musk, patchouli, balsamic essence, sandalwood oil, pine oil, cedar, and the like. Finished perfumes can comprise extremely complex mixtures of such ingredients. Finished perfumes may be included at a concentration ranging from about 0.01% to about 2% by weight of the detergent composition.

### Dye Transfer Inhibiting Agents

Dye transfer inhibiting agents are effective for inhibiting the transfer of dyes from one fabric to another during the cleaning process. Generally, such dye transfer inhibiting agents may include polyvinyl pyrrolidone polymers, polyamine N-oxide polymers, copolymers of N-vinylpyrrolidone and N-vinylimidazole, manganese phthalocyanine, peroxidases, and mixtures thereof. If used, these agents may be used at a concentration of about 0.0001% to about 10%, by weight of the composition, in some examples, from about 0.01% to about 5%, by weight of the composition, and in other examples, from about 0.05% to about 2% by weight of the composition.

### Chelating Agents

Preferably the composition comprises chelating agents and/or crystal growth inhibitor. Suitable molecules include copper, iron and/or manganese chelating agents and mixtures thereof. Suitable molecules include hydroxamic acids, aminocarboxylates, aminophosphonates, succinates, salts thereof, and mixtures thereof. Non-limiting examples of suitable chelants for use herein include ethylenediaminetetracetates, N-(hydroxyethyl)ethylenediaminetriacetates, nitrilotriacetates, ethylenediamine tetraproprionates, triethylenetetraaminehexacetates, diethylenetriamine-pentaacetates, ethanoldiglycines, ethylenediaminetetrakis (methylenephosphonates), diethylenetriamine penta(methylene phosphonic acid) (DTPMP), ethylenediamine disuccinate (EDDS), hydroxyethanedimethylenephosphonic acid (HEDP), methylglycinediacetic acid (MGDA), diethylenetriaminepentaacetic acid (DTPA), N,N-Dicarboxymethyl glutamic acid (GLDA) and salts thereof, and mixtures thereof. Other nonlimiting examples of chelants of use in the present disclosure are found in U.S. Pat. Nos. 7,445,644, 7,585,376 and 2009/0176684A1. Other suitable chelating agents for use herein are the commercial DEQUEST series, and chelants from Monsanto, DuPont, and Nalco, Inc. Yet other suitable chelants include the pyridinyl N Oxide type. Suitable chelants are disclosed in U.S. Pat. Nos. 9,550,964 and 8,841,247

### Suds Suppressors

Compounds for reducing or suppressing the formation of suds can be incorporated into the water-soluble unit dose articles. Suds suppression can be of particular importance in the so-called “high concentration cleaning process” and in front-loading style washing machines. Examples of suds supressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100 °C, silicone suds suppressors, and secondary alcohols.

Additional suitable antifoams are those derived from phenylpropylmethyl substituted polysiloxanes.

The detergent composition may comprise a suds suppressor selected from organomodified silicone polymers with aryl or alkylaryl substituents combined with silicone resin and a primary filler, which is modified silica. The detergent compositions may comprise from about 0.001% to about 4.0%, by weight of the composition, of such a suds suppressor.

The detergent composition comprises a suds suppressor selected from: a) mixtures of from about 80 to about 92% ethylmethyl, methyl(2-phenylpropyl) siloxane; from about 5 to about 14% MQ resin in octyl stearate; and from about 3 to about 7% modified silica; b) mixtures of from about 78 to about 92% ethylmethyl, methyl(2-phenylpropyl) siloxane; from about 3 to about 10% MQ resin in octyl stearate; from about 4 to about 12% modified silica; or c) mixtures thereof, where the percentages are by weight of the anti-foam.

### Suds Boosters

If high sudsing is desired, suds boosters such as the C₁₀-C₁₆ alkanolamides may be used. Some examples include the C₁₀-C₁₄ monoethanol and diethanol amides. If desired, water-soluble magnesium and/or calcium salts such as MgCl₂, MgSO₄, CaCl₂, CaSO₄, and the like, may be added at levels of about 0.1% to about 2% by weight of the detergent composition, to provide additional suds and to enhance grease removal performance.

### Conditioning Agents

Suitable conditioning agents include high melting point fatty compounds. The high melting point fatty compound useful herein has a melting point of 25 °C or higher, and is selected from the group consisting of fatty alcohols, fatty acids, fatty alcohol derivatives, fatty acid derivatives, and mixtures thereof. Suitable conditioning agents also include nonionic polymers and conditioning oils, such as hydrocarbon oils, polyolefins, and fatty esters.

Suitable conditioning agents include those conditioning agents characterized generally as silicones (e.g., silicone oils, polyoils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

### Fabric Enhancement Polymers

Suitable fabric enhancement polymers are typically cationically charged and/or have a high molecular weight. The fabric enhancement polymers may be a homopolymer or be formed from two or more types of monomers. The monomer weight of the polymer will generally be between 5,000 and 10,000,000, typically at least 10,000 and preferably in the range 100,000 to 2,000,000. Preferred fabric enhancement polymers will have cationic charge densities of at least 0.2 meq/gm, preferably at least 0.25 meq/gm, more preferably at least 0.3 meq/gm, but also preferably less than 5 meq/gm, more preferably less than 3 meq/gm, and most preferably less than 2 meq/gm at the pH of intended use of the composition, which pH will generally range from pH 3 to pH 9, preferably between pH 4 and pH 8. The fabric enhancement polymers may be of natural or synthetic origin.

### Pearlescent Agent

Non-limiting examples of pearlescent agents include: mica; titanium dioxide coated mica; bismuth oxychloride; fish scales; mono and diesters of alkylene glycol. The pearlescent agent may be ethyleneglycoldistearate (EGDS).

### Hygiene and Malodor

Suitable hygiene and malodor active agents include zinc ricinoleate, thymol, quaternary ammonium salts such as Bardac®, polyethylenimines (such as Lupasol® from BASF) and zinc complexes thereof, silver and silver compounds, especially those designed to slowly release Ag⁺ or nano-silver dispersions.

### Buffer System

The water-soluble unit dose articles described herein may be formulated such that, during use in aqueous cleaning operations, the wash water will have a pH of between about 7.0 and about 12, and in some examples, between about 7.0 and about 11. Techniques for controlling pH at recommended usage levels include the use of buffers, alkalis, or acids, and are well known to those skilled in the art. These include, but are not limited to, the use of sodium carbonate, citric acid or sodium citrate, lactic acid or lactate, monoethanol amine or other amines, boric acid or borates, and other pH-adjusting compounds well known in the art.

The detergent compositions herein may comprise dynamic in-wash pH profiles. Such detergent compositions may use wax-covered citric acid particles in conjunction with other pH control agents such that (i) about 3 minutes after contact with water, the pH of the wash liquor is greater than 10; (ii) about 10 minutes after contact with water, the pH of the wash liquor is less than 9.5; (iii) about 20 minutes after contact with water, the pH of the wash liquor is less than 9.0; and (iv) optionally, wherein, the equilibrium pH of the wash liquor is in the range of from about 7.0 to about 8.5.

### Method for Making

As exemplified by illustration in FIG. 3 of WO2020/023188 A1, a solution of a filament forming composition 35 is provided. The filament forming composition can comprise one or more filament forming materials and optionally one or more active agents. The filament forming composition 35 is passed through one or more die block assemblies 40 comprising a plurality of spinnerets 45 to form a plurality of fibrous elements 30 comprising the one or more filament forming materials and optionally one or more active agents. Multiple die block assemblies 40 can be employed to spin different layers of fibrous elements 30, with the fibrous elements 30 of different layers having a composition that differ from one another or are the same as one another. More than two die block assemblies in series can be provided to form three, four, or any other integer number of layers in a given ply. The fibrous elements 30 can be deposited on a belt 50 moving in a machine direction MD to form a first ply 10.

Particles can be introduced into the stream of the fibrous elements 30 between the die block assembly 40 and the belt 50. Particles can be fed from a particle receiver onto a belt feeder 41 or optionally a screw feeder. The belt feeder 41 can be set and controlled to deliver the desired mass of particles into the process. The belt feeder can feed an air knife 42 that suspends and directs the particles in an air stream into the fibrous elements 30 to form a particle-fiber layer of comingled fibrous elements 30 and particles that is subsequently deposited on the belt 50.

To form the water-soluble product, a first ply 10 can be provided. A second ply 15 can be provided separate from the first ply 10. The first ply 10 and the second ply 15 are superposed with one another. By superposed it is meant that one is positioned above or below the other with the proviso that additional plies or other materials, for example active agents, may be positioned between the superposed plies. A portion of the first ply 10 can be joined to a portion of the second ply 15 to form the water-soluble product 5. Each ply may comprise one or more layers.

### Particle-Fiber Layer

A particle-fiber layer may be arranged in several ways. Clusters of particles may be distributed in pockets distributed in the layer, where such pockets may be formed between layers of fibrous elements; the contact network and porosity within each cluster of particles is governed by physics of conventional particle packing, yet the clusters are substantially dilated in the layer.

The particles may be distributed relatively homogeneously throughout the fibrous structure, substantially free of local particle clusters; packing is substantially dilated on the scale of individual particles, with fewer inter-particle contacts and greater inter-particle porosity. Without wishing to be bound by theory, it is believed that a water-soluble unit dose article comprising a layer comprising fibrous elements and particles, where sticky surfactants, such as AES, are segregated into particles having a dilated structure, provides for an improvement in dispersion and dissolution of the unit dose article, both by faster imbibition of water into the dilated structure and by a reduction in contacts among particles having sticky surfactants.

When the solid article comprises a porous structure, the porous structure is can be made by one or more sheets, preferably each of said sheets is characterized by:
a Percent Open Cell Content of from 80% to 100%, preferably from 85% to 100%, more preferably from 90% to 100%; and/or
an Overall Average Pore Size of from 100 μm to 2000 μm, from 150 μm to 1000 μm, preferably from 200 μm to 600 μm; and/or
an Average Cell Wall Thickness of from 5 μm to 200 μm, preferably from 10 μm to 100 μm, more preferably from 10 μm to 80 μm; and/or
a final moisture content of from 0.5% to 25%, preferably from 1% to 20%, more preferably from 3% to 10%, by weight of said solid sheet article; and/or
a thickness of from 0.5 mm to 4 mm, preferably from 0.6 mm to 3.5 mm, more preferably from 0.7 mm to 3 mm, still more preferably from 0.8 mm to 2 mm, most preferably from 1 mm to 1.5 mm; and/or
a basis weight of from 50 grams/m2 to 250 grams/m2, preferably from 80 grams/m2 to 220 grams/m2, more preferably from 100 grams/m2 to 200 grams/m2; and/or
a density of from 0.05 grams/cm3 to 0.5 grams/cm3, preferably from 0.06 grams/cm3 to 0.4 grams/cm3, more preferably from 0.07 grams/cm3 to 0.2 grams/cm3, most preferably from 0.08 grams/cm3 to 0.15 grams/cm3; and/or
a Specific Surface Area of from 0.03 m2/g to 0.25 m2/g, preferably from 0.04 m2/g to 0.22 m2/g, more preferably from 0.05 m2/g to 0.2 m2/g, most preferably from 0.1 m2/g to 0.18 m2/g.

The physical characteristics of the porous solid sheets are described in WO 2020/147008 10 A1, pages 37 to 47.

### Method of Laundering

The present disclosure also encompasses a method of laundering using an article according to the present disclosure, comprising the steps of, placing at least one article according to the present disclosure into the washing machine along with the laundry to be washed, and carrying out a washing or cleaning operation.

Any suitable washing machine may be used. Those skilled in the art will recognize suitable machines for the relevant wash operation. The article of the present disclosure may be used in combination with other compositions, such as fabric additives, fabric softeners, rinse aids and the like.

The wash temperature may be 30 °C or less. The wash process may comprise at least one wash cycle having a duration of between 5 and 20 minutes. The automatic laundry machine may comprise a rotating drum, and wherein during at least one wash cycle, the drum has a rotational speed of between 15 and 40 rpm, preferably between 20 and 35 rpm.

The water-soluble unit dose article comprising a water-soluble fibrous or porous structure and one or more rheology-modified particles distributed throughout the structure may remove one or more types of proteinaceous stains across a broad wash pH profile.

### EXAMPLES

### Protease Stain Removal

Six detergent compositions (Compositions 1 to 6) were made and tested as detailed herein below.

### Test Method

### I. Preparation of Test Compositions

Tests were carried out using the following detergent compositions: Material additions shown as active material level in finished product (FP).

| | 1 | 2 | 3 | 4 | 5 | 6 |
|---|---|---|---|---|---|---|
| Ingredient | (g/L) | (g/L) | (g/L) | (g/L) | (g/L) | (g/L) |
| Sodium Alkyl Sulphate | 0.0429 | 0.0429 | 0.0429 | 0.0429 | 0.0429 | 0.0429 |
| PVOH 505¹ | 0.0587 | 0.0587 | 0.0587 | 0.0587 | 0.0587 | 0.0587 |
| Polyethylene oxide n10 | 0.0050 | 0.0050 | 0.0050 | 0.0050 | 0.0050 | 0.0050 |
| Polyethylene oxide n60k | 0.0008 | 0.0008 | 0.0008 | 0.0008 | 0.0008 | 0.0008 |
| NaLAS | 0.7345 | 0.7345 | 0.7345 | 0.7345 | 0.7345 | 0.7345 |
| Non-ionic surfactant | 0.0994 | 0.0994 | 0.0994 | 0.0994 | 0.0994 | 0.0994 |
| Trisodium salt of | 0.0256 | 0.0256 | 0.0256 | 0.0256 | 0.0256 | 0.0256 |
| methylglycine | | | | | | |
| diacetic acid | | | | | | |
| Polyacrylic Acid-Maleic | 0.0878 | 0.0878 | 0.0878 | 0.0878 | 0.0878 | 0.0878 |
| Acid Co-Polymer² | | | | | | |
| Serine protease 1³ | 0.0000 | 0.0099 | 0.0000 | 0.0000 | 0.0099 | 0.0099 |
| Metalloprotease 1⁴ | 0.0000 | 0.0000 | 0.0826 | 0.0000 | 0.0826 | 0.0000 |
| Metalloprotease 2⁵ | 0.0000 | 0.0000 | 0.0000 | 0.0118 | 0.0000 | 0.0118 |
| Process aids, Perfume, | balance | balance | balance | balance | balance | balance |
| Misc. & Moisture | | | | | | |

| | |
|---|---|
| NaOH | As required to achieve pH 7/8/9/or 10 |

¹Celvol PVOH 505 Mw 40,000-50,000 g/mol, 72-75% hydrolyzed, available from Kuraray America
²Copolymer 50% acrylic acid, 50% maleic acid
³Protease variant having more than 90% identity to SEQ ID No: 2 @ 156.8 mg/g
⁴Neutrase from Sigma Aldrich @ 18.2 mg/g
⁵SEQ ID NO: 33 of WO2014/194034@ 44.7 mg/g

### II. Test Procedure

| Wash Equipment | Automated Tergotometer | | |
|---|---|---|---|
| Wash Volume | 1000 | ml | |
| Water Hardness | Tap water | | ~7 gpg hardness |
| Water Temperature | 25 | °C | |
| Detergent addition | 0.75 | g | Detergent + enzymes ‘on top’ |

| | | |
|---|---|---|
| Protease stains | POM = Pigment oil milk | Centre for text materials B.V., |
| | CMCB = Chocolate milk & carbon | Het Scheur, Stoomloggerweg |
| | black | 11, 3133 KT Vlaardingen, |
| | CSM = Choc soy milk | Netherlands |
| Reps of stains | 2 internal reps | General Laboratory (with trap) |
| | 2 external reps | 3-piece, glass, 9 cm diameter |
| | | 1 L + Pyrex with side-arm. |
| Test method | 12 min wash | 25 °C |
| | 1 × 5 mins rinse | 15 °C |
| Measure of stain | Image analysis | VeriVide - Digieye system for |
| removal SRI | | textiles |

### Analysis of Protease Stain Removal on Fabric

Determination of protease stain removal on fabric was completed using the following procedure. Two external replicates of each stain were completed for each test product. 1000 ml of tap water was measured into each tergotometer pot at 25 °C. Dilute NaOH solution was then used to alter the pH of the water to pH 7, 8, 9 or 10. Each pH was tested for each of the products 1-6 above. 0.75 g of the detergent was then added to each pot and for legs 2/3/4 the required enzymes were added on top. The stains were washed for 12 min wash at 25 °C followed by a 5 min rinse at 15 °C.

At the start of the test the stains were all pre-measured before washing using Digi eye. And they were measured again after washing

Results below show the measured level of stain removal per product per pH by SRI calculated as:

SRI=100*(ΔEb−ΔEa)/ΔEb

ΔEb=SQRT(L\hat{ }2+a\hat{ }2+b\hat{ }2)before washing

ΔEa=SQRT(L\hat{ }2+a\hat{ }2+b\hat{ }2) after washing

pH 7 results

| | | | | | delta | | | delta | | |
|---|---|---|---|---|---|---|---|---|---|---|
| Composition | 1 | | 2 | | vs 1 | 3 | | vs 1 | 4 | |
| Overall SRI | 18.51 | stdev | 23.59 | stdev | 5.08 | 21.01 | stdev | 2.50 | 34.87 | stdev |
| POM | 24.432 | 1.410 | 27.671 | 0.286 | 3.24 | 25.351 | 2.344 | 0.92 | 39.221 | 4.764 |
| CMCB | 15.439 | 1.870 | 22.095 | 2.410 | 6.66 | 20.303 | 1.321 | 4.86 | 30.389 | 4.773 |
| CSM | 15.667 | 0.842 | 21.003 | 0.885 | 5.34 | 17.387 | 2.756 | 1.72 | 35.006 | 4.983 |

pH 7 results continued

| | delta | | | delta | | | delta |
|---|---|---|---|---|---|---|---|
| Composition | vs 1 | 5 | | vs 1 | 6 | | vs 1 |
| Overall SRI | 16.36 | 22.86 | stdev | 4.35 | 35.25 | stdev | 16.73 |
| POM | 14.79 | 27.621 | 2.749 | 3.19 | 40.161 | 5.882 | 15.73 |
| CMCB | 14.95 | 20.556 | 1.196 | 5.12 | 30.718 | 2.434 | 15.28 |
| CSM | 19.34 | 20.413 | 3.289 | 4.75 | 34.860 | 0.864 | 19.19 |

pH 8 results

| | | | | | delta | | | delta | | |
|---|---|---|---|---|---|---|---|---|---|---|
| Composition | 1 | | 2 | | vs 1 | 3 | | vs 1 | 4 | |
| Overall SRI | 18.69 | stdev | 26.80 | stdev | 8.11 | 18.90 | stdev | 0.21 | 34.02 | stdev |
| POM | 24.368 | 1.332 | 30.758 | 1.504 | 6.39 | 25.940 | 2.778 | 1.57 | 40.068 | 4.018 |
| CMCB | 17.474 | 1.833 | 25.667 | 2.155 | 8.19 | 17.465 | 1.526 | −0.01 | 27.853 | 5.758 |
| CSM | 14.218 | 1.868 | 23.975 | 1.350 | 9.76 | 13.283 | 2.881 | −0.93 | 34.130 | 3.722 |

Ph 8 Results continued

| | delta | | | delta | | | delta |
|---|---|---|---|---|---|---|---|
| Composition | vs 1 | 5 | | vs 1 | 6 | | vs 1 |
| Overall SRI | 15.33 | 25.99 | stdev | 7.31 | 36.17 | stdev | 17.48 |
| POM | 15.70 | 29.726 | 1.677 | 5.36 | 42.418 | 7.327 | 18.05 |
| CMCB | 10.38 | 23.360 | 4.042 | 5.89 | 31.810 | 5.889 | 14.34 |
| CSM | 19.91 | 24.896 | 1.614 | 10.68 | 34.272 | 4.255 | 20.05 |

pH 9 results

| | | | | | delta | | | delta | | |
|---|---|---|---|---|---|---|---|---|---|---|
| Composition | 1 | | 2 | | vs 1 | 3 | | vs 1 | 4 | |
| Overall SRI | 18.02 | stdev | 30.51 | stdev | 12.49 | 18.38 | stdev | 0.36 | 30.82 | stdev |
| POM | 27.209 | 2.914 | 35.428 | 1.120 | 8.22 | 24.822 | 0.763 | −2.39 | 37.992 | 3.794 |
| CMCB | 16.786 | 0.651 | 27.824 | 1.326 | 11.04 | 18.055 | 1.781 | 1.27 | 26.748 | 2.554 |
| CSM | 10.069 | 2.611 | 28.280 | 3.465 | 18.21 | 12.267 | 1.764 | 2.20 | 27.710 | 5.503 |

pH 9 results continued

| | delta | | | delta | | | delta |
|---|---|---|---|---|---|---|---|
| Composition | vs 1 | 5 | | vs 1 | 6 | | vs 1 |
| Overall SRI | 12.80 | 30.85 | stdev | 12.83 | 40.14 | stdev | 22.12 |
| POM | 10.78 | 35.447 | 2.520 | 8.24 | 48.701 | 8.727 | 21.49 |
| CMCB | 9.96 | 28.374 | 2.315 | 11.59 | 36.626 | 7.535 | 19.84 |
| CSM | 17.64 | 28.721 | 2.994 | 18.65 | 35.087 | 4.718 | 25.02 |

pH 10 results

| | | | | | delta | | | delta | | |
|---|---|---|---|---|---|---|---|---|---|---|
| Composition | 1 | | 2 | | vs 1 | 3 | | vs 1 | 4 | |
| Overall SRI | 16.24 | stdev | 41.19 | stdev | 24.96 | 17.81 | stdev | 1.57 | 19.54 | stdev |
| POM | 26.225 | 0.626 | 51.404 | 2.474 | 25.18 | 28.154 | 0.960 | 1.93 | 28.942 | 4.555 |
| CMCB | 15.789 | 1.407 | 39.048 | 6.861 | 23.26 | 17.560 | 2.003 | 1.77 | 18.504 | 2.505 |
| CSM | 6.700 | 0.914 | 33.133 | 6.194 | 26.43 | 7.704 | 4.973 | 1.00 | 11.168 | 3.019 |

pH 10 results continued

| | delta | | | delta | | | delta |
|---|---|---|---|---|---|---|---|
| Composition | vs 1 | 5 | | vs 1 | 6 | | vs 1 |
| Overall SRI | 3.30 | 41.72 | stdev | 25.48 | 43.80 | stdev | 27.56 |
| POM | 2.72 | 50.862 | 7.165 | 24.64 | 55.645 | 3.907 | 29.42 |
| CMCB | 2.72 | 40.478 | 11.982 | 24.69 | 40.885 | 6.932 | 25.10 |
| CSM | 4.47 | 33.823 | 5.570 | 27.12 | 34.868 | 1.872 | 28.17 |

### Overall SRI Across pH Summary:

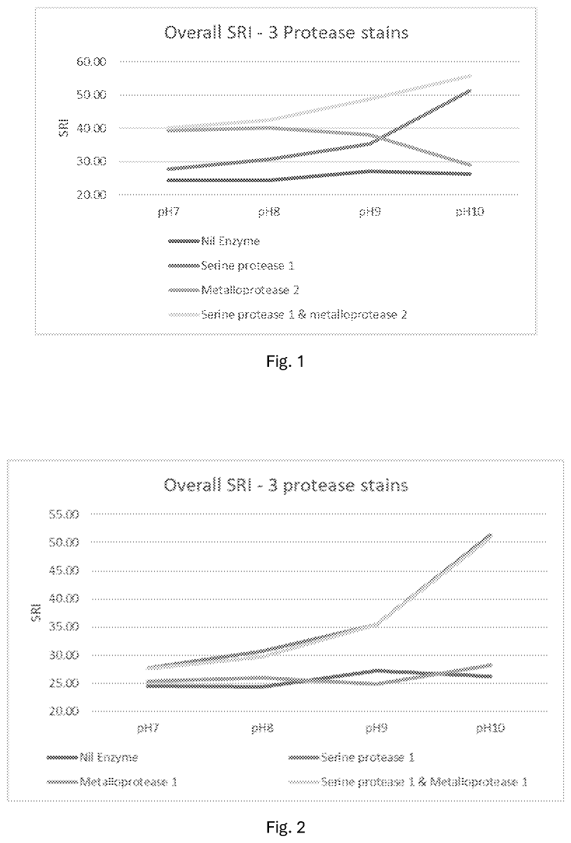

Results are shown in FIGS. 1 and 2.

The examples demonstrate the efficacy of a serine protease and metalloprotease 2 (SEQ ID NO: 33 of WO2014/194034) enzyme system within solid unit dose to deliver robust protease stain removal across pH 7 to 10.

As used in this specification and the claims that follow, the articles “a”, “an”, and “the” include singular and plural references unless the context clearly dictates otherwise. As such, the terms “a” or “an”, “one or more” and “at least one” can be used interchangeably herein. Thus, for example, “a component” may include one or more components unless the reference is specifically indicated as being singular.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

For clarity purposes, the total “% wt” values do not exceed 100% wt.

Every document cited herein, including any cross referenced or related patent or application, is hereby incorporated herein by reference in its entirety unless expressly excluded or otherwise limited. The citation of any document is not an admission that it is prior art with respect to any product or method disclosed or claimed herein or that it alone, or in any combination with any other reference or references, teaches, suggests or discloses any such product or method. Further, to the extent that any meaning or definition of a term in this document conflicts with any meaning or definition of the same term in a document incorporated by reference, the meaning or definition assigned to that term in this document shall govern.

While particular examples and/or embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A water-soluble unit-dose solid detergent article comprising a porous or a fibrous structure, the article comprising
a surfactant system
a serine protease; and
a metalloprotease variant, the variant having a sequence identity of at least about 60% to the metalloprotease of SEQ ID NO:1.

2. The solid detergent article according to claim 1, wherein the serine protease is a variant, wherein said variant has at least about 90% amino acid identity with Bacillus lentus subtilisin GG36 protease comprising the amino acid sequence shown in SEQ ID NO:2 and said variant comprises an amino acid sequence comprising one of the combinations of amino acid substitutions: T022A-S103A-V104IN116L-G159D-S188D-A232V-Q245R-N248D-E271F, T022A-S101G-V104I-N116L-G159D-S188D-A232V-Q245R-N248D-E271F, T022A-S101G-S103A-V104I-N116L-S188D-A232V-Q245R-N248D-E271F, T022A-S101G-S103A-V104I-N116L-G159D-S188D-Q245R-N248D-E271F, T022A-S101G-S103A-V104I-N116L-G159D-S188D-A232V-N248D-E271F, T022A-S101G-S103A-V104I-N116L-G159D-S188D-A232V-Q245R-E271F, T022A-S101G-S103A-V104I-N116L-G159D-S188D-A232V-Q245R-N248D-E271F, T022A-T033S-S101G-S103A-V104I-N116L-G159D-S188D-T213A-A232V-Q245R-N248D-E271F, T022A-V068A-S101G-S103A-V104IN116L-G159D-S188D-T213A-A232V-Q245R-N248D-E271F, T022A-S101G-S103A-V104IN116L-G159D-S188D-T213A-A232V-Q245R-N248D-E271F, T022A-S101G-S103A-V104IN116L-S128L-G159D-S188D-A232V-Q245R-N248D-E271F, T022A-V068A-S101G-S103A-V104I-N116L-G159D-S188D-A232V-Q245R-N248D-E271F, T022A-S101G-S103A-V104I-N116L-G159D-S188D-A232V-Q245R-N248D, T022A-S101G-S103A-V104I-N116L-S128L-G159D-S188D-T213A-A232V-Q245R-N248D-E271F, T022A-N062D-S101G-S103A-V104I-N116L-G159D-S188D-T213A-A232V-Q245R-N248D-E271F, T022A-T033S-S101G-S103A-V104I-N116L-S128L-G159D-S188D-A232V-Q245R-N248D-E271F, T022A-T033S-S101G-S103A-V104I-N116L-G159D-S188D-A232V-Q245R-N248D-E271F, T022A-T033S-S101G-S103A-V104I-N116L-S128L-G159D-S188D-T213A-A232V-Q245R-N248D-E271F, wherein amino acid positions of the subtilisin variant are numbered according to the numbering of corresponding amino acid positions in the amino acid sequence of Bacillus amyloliquefaciens subtilisin BPN′ shown in SEQ ID NO:3, and wherein amino acid positions of the subtilisin variant are numbered according to the numbering of corresponding amino acid positions in the amino acid sequence of Bacillus amyloliquefaciens subtilisin BPN′ shown in SEQ ID NO:3.

3. The solid detergent article according to claim 1, wherein the solid detergent article comprises from about 10 to about 90% by weight of the article of the surfactant system and wherein the surfactant system comprises one or more surfactants selected from the group consisting of anionic, non-ionic, amphoteric and, a mixture thereof.

4. The solid detergent article according to claim 1, wherein the solid detergent article comprises a surfactant system and the surfactant system comprises an anionic surfactant selected from the group consisting of alkylbenzene sulfonate, alkyl sulfate, alkyl alkoxylate sulfate, and a mixture thereof.

5. The solid detergent article according to claim 1, wherein the solid detergent article comprises a surfactant system and the surfactant system comprises linear alkylbenzene sulfonate and alkyl alkoxylate sulfate in a weight ratio of linear alkylbenzene sulfonate to alkyl alkoxylate sulfate of greater than 1.

6. The solid detergent article according to claim 1, wherein the solid detergent article comprises a surfactant system and the surfactant system comprises amino oxide and alkyl alkoxylate sulfate in a weight ratio of from about 8:1 to about 1:1.

7. The solid detergent article according to claim 1, wherein the solid detergent article comprises from about 0.5 to about 10% by weight of the article of C₆-C₂₀ linear or branched alkylalkoxylated alcohols (AA) having a weight average degree of alkoxylation ranging from about 5 to about 15, and combinations thereof.

8. The solid detergent article according to claim 1 further comprising an additional enzyme selected from the group consisting of proteases, amylases, cellulases, lipases, xyloglucanases, mannanases, nucleases, pectate lyases, perhydrolases, and a mixture thereof.

9. The solid detergent article according to claim 1, wherein the serine protease and/or the metalloprotease and/or other enzymes, if present, are incorporated within the article as a prill, a spray on slurry, or a combination thereof.

10. The solid detergent article according to claim 1, further comprising a water-soluble polymer, the water-soluble polymer is selected from polyvinyl alcohol, polyethylene glycol, polysaccharides, and a mixture thereof.

11. The solid detergent article according to claim 1 comprising from about 5 to about 20% by weight of the article of polyvinyl alcohol.

12. The solid detergent article according to claim 1, further comprising an inorganic dissolution aid, a rheology modifier, or a combination thereof.

13. The solid detergent article according to claim 1, further comprising a perfume microcapsule, a hueing agent, a bleaching agent, an aversive agent, a nonionic surfactant, or a combination thereof.

14. The solid detergent article according to claim 1, wherein the solid detergent article has a pH of from about 7 to about 10 as measured in a 10% by weight demineralized water solution at 20 °C.

15. The solid detergent article according to claim 1, wherein the article comprises one or more particles.

16. The solid detergent article according to claim 1, wherein the water-soluble unit-dose solid detergent article comprises a fibrous structure wherein said soluble fibrous structure forms a pouch.

17. The solid detergent article according to claim 1, wherein the water-soluble unit-dose solid detergent article comprises a porous structure comprising one or more sheets wherein each sheet is characterized by a Percent Open Cell Content of from about 80% to about 100% and an Overall Average Pore Size of from about 100 μm to about 2000 μm.

18. The solid detergent article according to claim 1, wherein the water-soluble unit-dose solid detergent article comprises a porous structure comprising one or more sheets wherein said one or more sheet is characterized by:
a Percent Open Cell Content of from about 90% to about 100%; and/or
an Overall Average Pore Size of from about 200 μm to about 600 μm; and/or
an Average Cell Wall Thickness of from about 10 μm to about 80 μm; and/or
a final moisture content of from about 3% to about 10%, by weight of said solid sheet article; and/or
a thickness of from about 1 mm to about 1.5 mm; and/or
a basis weight of from about 100 grams/m2 to about 200 grams/m2; and/or
a density of from about 0.05 grams/cm3 to about 0.5 grams/cm3; and/or
a Specific Surface Area of from about 0.03 m2/g to about 0.25 m2/g.

19. A method of cleaning a surface, the method comprising the step of adding the solid detergent article according to claim 1 to water to create a wash liquor, subjecting the surface to the wash liquor and rinsing the surface and wherein the surface is a fabric.
